SEQUENCE LISTING (26 sequences)

SEQ ID NO: 1 - DNA, 5022 bp, Homo sapiens
  CDS (1)..(5022)
atggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaagaccccaaaaaactaaatcacactcctgtatcaaccatgagttcttctcagcctgtgtcacgaccattgcaacccatacaaccagcaccgcctcttcaaccatctggggtgccaacaagtggaccatctcagaccaccatacacttactacctacagctccaactaccgtgaatgtaacacatcgtccagtaactcaggtgaccacaagactccctgtaccaagagctcctgcaaaccaccaggtggtttatacaactcttcctgcaccaccagctcaggctcccttgcgaggaactgttatgcaggctcctgctgttcggcaggtcaatccccaaaataccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtag

SEQ ID NO: 2 - protein, 1673 aa, Homo sapiens
MDSLEEPQKKVFKARKTMRVSDRQQLEAVYKVKEELLKTDVKLLNGNHENGDLDPTSPLENMDYIKDKEEVNGIEEICFDPEGSKAEWKETPCILSVNVKNKQDDDLNCEPLSPHNITPEPVSKLPAEPVSGDPAPGDLDAGDPASGVLASGDSTSGDPTSSEPSSSDAASGDATSGDAPSGDVSPGDATSGDATADDLSSGDPTSSDPIPGEPVPVEPISGDCAADDIASSEITSVDLASGAPASTDPASDDLASGDLSSSELASDDLATGELASDELTSESTFDRTFEPKSVPVCEPVPEIDNIEPSSNKDDDFLEKNGADEKLEQIQSKDSLDEKNKADNNIDANEETLETDDTTICSDRPPENEKKVEEDIITELALGEDAISSSMEIDQGEKNEDETSADLVETINENVIEDNKSENILENTDSMETDEIIPILEKLAPSEDELTCFSKTSLLPIDETNPDLEEKMESSFGSPSKQESSESLPKEAFLVLSDEEDISGEKDESEVISQNETCSPAEVESNEKDNKPEEEEQVIHEDDERPSEKNEFSRRKRSKSEDMDNVQSKRRRYMEEEYEAEFQVKITAKGDINQKLQKVIQWLLEEKLCALQCAVFDKTLAELKTRVEKIECNKRHKTVLTELQAKIARLTKRFEAAKEDLKKRHEHPPNPPVSPGKTVNDVNSNNNMSYRNAGTVRQMLESKRNVSESAPPSFQTPVNTVSSTNLVTPPAVVSSQPKLQTPVTSGSLTATSVLPAPNTATVVATTQVPSGNPQPTISLQPLPVILHVPVAVSSQPQLLQSHPGTLVTNQPSGNVEFISVQSPPTVSGLTKNPVSLPSLPNPTKPNNVPSVPSPSIQRNPTASAAPLGTTLAVQAVPTAHSIVQATRTSLPTVGPSGLYSPSTNRGPIQMKIPISAFSTSSAAEQNSNTTPRIENQTNKTIDASVSKKAADSTSQCGKATGSDSSGVIDLTMDDEESGASQDPKKLNHTPVSTMSSSQPVSRPLQPIQPAPPLQPSGVPTSGPSQTTIHLLPTAPTTVNVTHRPVTQVTTRLPVPRAPANHQVVYTTLPAPPAQAPLRGTVMQAPAVRQVNPQNTLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 3 - DNA, 5046 bp, Homo sapiens
  CDS (1)..(5046)
atgcatcaggaccagagattcagaatggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaagaccccaaaaaactaaatcacactcctgtatcaaccatgagttcttctcagcctgtgtcacgaccattgcaacccatacaaccagcaccgcctcttcaaccatctggggtgccaacaagtggaccatctcagaccaccatacacttactacctacagctccaactaccgtgaatgtaacacatcgtccagtaactcaggtgaccacaagactccctgtaccaagagctcctgcaaaccaccaggtggtttatacaactcttcctgcaccaccagctcaggctcccttgcgaggaactgttatgcaggctcctgctgttcggcaggtcaatccccaaaataccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtag

SEQ ID NO: 4 - protein, 1681 aa, Homo sapiens [sequence omitted]

SEQ ID NO: 5 - DNA, 4661 bp, Homo sapiens
  CDS (159)..(3971)
tttttgaatctgcggaggcggcggcggtggcagcggcggcgcggcgactgaagcgcgcgaaaagctgaggcggcaacgtcggggacggctgcgcgggacggctctgtaggaaggaacttggttccccctccctcagcttccgccccaaaagattcagaatggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaagaccccaaaaaactaaatcacactcctgtatcaaccatgagttcttctcagcctgtgtcacgaccattgcaacccatacaaccagcaccgcctcttcaaccatctggggtgccaacaagtggaccatctcagaccaccatacacttactacctacagctccaactaccgtgaatgtaacacatcgtccagtaactcaggtgaccacaagactccctgtaccaagagctcctgcaaaccaccaggtggtttatacaactcttcctgcaccaccagctcaggctcccttgcgaggaactgttatgcaggctcctgctgttcggcaggtcaatccccaaaatagtgttacagttcgagtgcctcaaacaaccacatatgttgtaaacaatggactaaccctgggatcaacaggacctcagctcacagtgcatcaccgaccaccacaagtgcatactgagcccccacgccccgtgcacccagcacccttaccagaagctccacaaccacagcgtctgcccccagaagctgccagcacatctctgcctcagaagccacacttgaagttagcacgcgttcagagtcaaaatggcatagtactgtcatggagtgtcctggaggtggatcgaagctgtgccactgttgatagctaccatctctatgcttaccatgaggaacccagtgccactgtgccctcacaatggaaaaagattggggaagtcaaggcacttcccttgcccatggcatgtactctcacccagtttgtatctggtagcaaatactactttgcagtacgagccaaggatatttatggacgttttgggcctttctgtgatcctcagtcaacagatgtgatctcttctacccagagcagttaaaccttggagcctttatattttcctcttttaaaatttccaccttttggtcttgtttttaatcttgtgcatgataccccatgtaaaatccaccttgtgcaagatttcttggacagatgtgtgtatacactacatttgtttataaccagaagcaaaataaactcagcccacaaagctagaatcttttcctggacagtttaggctttggggtttggaaatgtaaatgtgtaccttgctttagttttgaggctggggaatatgtgtgggtgtttatgtgtgtttttccttatgtaggtgttattgcattggagtctcccattttcattctcaaatttacctcttaaagtacgaagtaagtagatcaaaggatttgagatgtgtaactggcatgattctgcttttgaaggatctatagtatcattttagttaagtgggtcaaacagaatcaaaacaaaacccaaagaaataaataaaaaacaaaatggctaaatagtttaaaataggttaattcgaacacaggaaaggatctatttgttgtttcttttgtctggtctcctgagttgttaattaggtgaaaaaagatctgcaatggccccctccctttcctaatctggcttttacatttattttgtgccttaaagattaactacaaagataaacatggccaaaaataaataaataaatatggccata

SEQ ID NO: 6 - protein, 1270 aa, Homo sapiens
MDSLEEPQKKVFKARKTMRVSDRQQLEAVYKVKEELLKTDVKLLNGNHENGDLDPTSPLENMDYIKDKEEVNGIEEICFDPEGSKAEWKETPCILSVNVKNKQDDDLNCEPLSPHNITPEPVSKLPAEPVSGDPAPGDLDAGDPASGVLASGDSTSGDPTSSEPSSSDAASGDATSGDAPSGDVSPGDATSGDATADDLSSGDPTSSDPIPGEPVPVEPISGDCAADDIASSEITSVDLASGAPASTDPASDDLASGDLSSSELASDDLATGELASDELTSESTFDRTFEPKSVPVCEPVPEIDNIEPSSNKDDDFLEKNGADEKLEQIQSKDSLDEKNKADNNIDANEETLETDDTTICSDRPPENEKKVEEDIITELALGEDAISSSMEIDQGEKNEDETSADLVETINENVIEDNKSENILENTDSMETDEIIPILEKLAPSEDELTCFSKTSLLPIDETNPDLEEKMESSFGSPSKQESSESLPKEAFLVLSDEEDISGEKDESEVISQNETCSPAEVESNEKDNKPEEEEQVIHEDDERPSEKNEFSRRKRSKSEDMDNVQSKRRRYMEEEYEAEFQVKITAKGDINQKLQKVIQWLLEEKLCALQCAVFDKTLAELKTRVEKIECNKRHKTVLTELQAKIARLTKRFEAAKEDLKKRHEHPPNPPVSPGKTVNDVNSNNNMSYRNAGTVRQMLESKRNVSESAPPSFQTPVNTVSSTNLVTPPAVVSSQPKLQTPVTSGSLTATSVLPAPNTATVVATTQVPSGNPQPTISLQPLPVILHVPVAVSSQPQLLQSHPGTLVTNQPSGNVEFISVQSPPTVSGLTKNPVSLPSLPNPTKPNNVPSVPSPSIQRNPTASAAPLGTTLAVQAVPTAHSIVQATRTSLPTVGPSGLYSPSTNRGPIQMKIPISAFSTSSAAEQNSNTTPRIENQTNKTIDASVSKKAADSTSQCGKATGSDSSGVIDLTMDDEESGASQDPKKLNHTPVSTMSSSQPVSRPLQPIQPAPPLQPSGVPTSGPSQTTIHLLPTAPTTVNVTHRPVTQVTTRLPVPRAPANHQVVYTTLPAPPAQAPLRGTVMQAPAVRQVNPQNSVTVRVPQTTTYVVNNGLTLGSTGPQLTVHHRPPQVHTEPPRPVHPAPLPEAPQPQRLPPEAASTSLPQKPHLKLARVQSQNGIVLSWSVLEVDRSCATVDSYHLYAYHEEPSATVPSQWKKIGEVKALPLPMACTLTQFVSGSKYYFAVRAKDIYGRFGPFCDPQSTDVISSTQSS

SEQ ID NO: 7 - DNA, 9019 bp, Homo sapiens
  CDS (321)..(4157)
gacgggattcacaggcattatgttacaaaactggcttgctgccttctgttttctttttctgtgtcctgtgtagctgcattgtttgttgcagtatatgcattagctgttgtctatgttcagtttcctgcaggctgacaaagtgggaggggctttgtttgttacatagctgagttttcactgtgtgagttcttgagtgcacattgcagccatcttaattacacttttgtgcaactgagactgtgcagctgtctcaaaatggctgctaacctagtctttgtagctgcttaggcagcaagtatgtcagtgaggagacttgtcacatgcatcaggaccagagattcagaatggacagtttagaagaacctcagaaaaaagtctttaaggctcgaaaaacgatgagagtgagtgatcgtcagcaacttgaagcagtgtacaaggtcaaagaagaactgttgaaaactgatgtcaagctgttaaatggcaaccatgaaaatggagatttggacccaacctcacctttggaaaacatggattacattaaagacaaggaagaggtgaatggcattgaagagatttgttttgatcctgaaggaagtaaagcagaatggaaggaaacaccctgtatcctaagtgttaatgtaaaaaacaagcaggatgatgatttaaattgtgaacctttgtctccccataatataactccagaaccagtctctaaactgcctgctgaaccagtttctggtgatccagcccctggtgatctggatgccggagatccagcctccggagtactggcctctggtgattccacctctggtgatcccacctctagcgagccctcctctagtgatgctgcctctggtgatgcaacctctggtgatgccccttctggtgatgtgtcccctggtgatgccacctctggtgatgccactgctgatgatctctcctctggtgatcccacctctagtgatcccatcccaggtgaaccggtccctgttgaacccatttctggtgattgtgccgctgatgatatagcctctagtgaaataacttctgttgatctggcttctggagcaccagcttccactgatccagcctctgatgatctggcctctggtgatctatcctctagtgaactggcctctgatgatctggccactggtgaactggcctctgatgagctgacttctgaatcaacctttgatcgtacctttgaaccaaagtctgtaccagtttgtgaaccagttcctgaaattgacaatatagaaccaagtagcaataaagatgatgattttcttgaaaaaaatggagctgatgaaaaattagagcaaattcagagtaaagactcattggatgagaaaaataaagctgataataatattgatgctaatgaagaaactctagaaacagatgatacaactatttgttcagatcgacctcctgaaaatgaaaagaaggtagaggaagatattatcacagagcttgctcttggagaagatgctatatctagcagtatggaaattgaccaaggtgaaaagaatgaagatgaaacttctgcagatcttgtagaaacgattaatgaaaatgttattgaagataacaaaagtgagaatatcttagaaaatacagactctatggagacagatgaaatcattcctattttggaaaagcttgcaccttctgaggatgaacttacttgcttttctaaaacatctctccttccaatcgatgagacaaatccagatttggaagagaaaatggaaagttcttttggttcaccatctaaacaagaaagtagtgagagtttgccaaaagaagcctttctggtcctctctgatgaagaggatatttcgggtgaaaaagatgagtctgaagttatatcgcaaaatgaaacgtgctctccagcagaagtagaaagtaatgaaaaggacaacaaacctgaggaagaagagcaagtaatacatgaagatgatgaaagaccttctgagaaaaatgaattttctagacgaaaacgttctaaatcagaagacatggacaatgtacagtctaaacgtcgtcgatatatggaagaagaatatgaggcagaatttcaagtaaagattacagccaaaggagacattaaccagaaacttcaaaaggttatacagtggttgctggaagaaaaattgtgtgcgctgcagtgtgctgtatttgataagactttggcagaattgaaaacacgagtggaaaagattgaatgtaacaagaggcataaaacagttctcactgaactacaggccaagatagccaggttaaccaaacgctttgaagcagccaaagaagatcttaagaaaagacatgaacatccacccaacccaccagtatcaccaggaaaaactgtaaatgatgtcaacagcaataataacatgtcttacagaaatgcaggcacagtgagacagatgctggagtccaaaagaaatgtaagcgagagtgcaccaccatcctttcaaactcctgtgaatacagtatcttcaaccaatcttgtcactcctccagcagttgtcagtagtcaacctaaattgcagactccagtgacttcgggttccctcacagcaacgtcagttcttcctgcacccaatacagctactgtagttgctactactcaggtgcctagtggaaatccccagcctacaatctctttacagcctttgccagtgattttgcatgtacctgttgcagtatcctcccagcctcagcttctacagagccatccagggactttggtgactaatcaaccatctggcaatgttgaattcatttctgtgcaaagcccacctacagtgagtggtcttaccaaaaatccagtatccttgccatccttgccaaatcccactaaaccaaacaacgttccttctgtgcccagtcctagtattcaaaggaaccctactgccagtgctgcaccattgggaacaacacttgctgtgcaggctgttccaacagcacactctattgtacaagccacaaggacttctttacccacagtgggcccatcaggactctatagtccatcaactaatcgaggtcctatacagatgaaaattccaatttctgcatttagtacttcgtctgctgcagaacagaacagcaataccaccccaagaattgaaaaccagacaaacaaaacaatagatgcttctgtcagtaagaaagcagctgatagcacatcacagtgtggaaaagccactggcagtgattcaagtggtgtcattgatctcacaatggatgatgaagagagtggagcttcacaagaccccaaaaaactaaatcacactcctgtatcaaccatgagttcttctcagcctgtgtcacgaccattgcaacccatacaaccagcaccgcctcttcaaccatctggggtgccaacaagtggaccatctcagaccaccatacacttactacctacagctccaactaccgtgaatgtaacacatcgtccagtaactcaggtgaccacaagactccctgtaccaagagctcctgcaaaccaccaggtggtttatacaactcttcctgcaccaccagctcaggctcccttgcgaggaactgttatgcaggctcctgctgttcggcaggtcaatccccaaaatagtgttacagttcgagtgcctcaaacaaccacatatgttgtaaacaatggactaaccctgggatcaacaggacctcagctcacagtgcatcaccgaccaccacaagtgcatactgagcccccacgccccgtgcacccagcacccttaccagaagctccacaaccacagcgtctgcccccagaagctgccagcacatctctgcctcagaagccacacttgaagttagcacgcgttcagagtcaaaatggcatagtactgtcatggagtgtcctggaggtggatcgaagctgtgccactgttgatagctaccatctctatgcttaccatgaggaacccagtgccactgtgccctcacaatggaaaaagattggggaagtcaaggcacttcccttgcccatggcatgtactctcacccagtttgtatctggtagcaaatactactttgcagtacgagccaaggatatttatggacgttttgggcctttctgtgatcctcagtcaacagatgtgatctcttctacccagagcagttaaaccttggagcctttatattttcctcttttaaaatttccaccttttggtcttgtttttaatcttgtgcatgataccccatgtaaaatccaccttgtgcaagatttcttggacagatgtgtgtatacactacatttgtttataaccagaagcaaaataaactcagcccacaaagctagaatcttttcctggacagtttaggctttggggtttggaaatgtaaatgtgtaccttgctttagttttgaggctggggaatatgtgtgggtgtttatgtgtgtttttccttatgtaggtgttattgcattggagtctcccattttcattctcaaatttacctcttaaagtacgaagtaagtagatcaaaggatttgagatgtgtaactggcatgattctgcttttgaaggatctatagtatcattttagttaagtgggtcaaacagaatcaaaacaaaacccaaagaaataaataaaaaacaaaatggctaaatagtttaaaataggttaattcgaacacaggaaaggatctatttgttgtttcttttgtctggtctcctgagttgttaattaggtgaaaaaagatctgcaatggccccctccctttcctaatctggcttttacatttattttgtgccttaaagattaactacaaagataaacatggccaaaaataaataaataaatatggccatatgtccgttgttgcttagtcttcccttgcagccttttacccttgatttctccttcatctctaccaaatatagcacaactcctcaagtaattttttttttttaagatggagttttgctcttgttgcccaggctagagtgcggtggcacaatcttggctcactgcaacctctgcctcctgggttcaagcaattcttctgtctcagccccagagtagctgggattacaggcacctgtcaccatgcccggctaatttttgtatttttagtagagatggggtttcaccatgttggccaggctggtcttgaactcctgacctcaggtgatccacccgcctcggcctcccagagtgctgcgattacagttgtgagccactgtatccagcctactcaagtgatttttaaaccaaggtgtgtgtatgtacatggatgtgtatgtacacacacacacaggtgcatgcatagtctcatcttagccgggcatggtggcgacaatcacatttaactccaaaaatttgacgtgctattttcttctaaaactatagatgcttttatttttggtcactatttaactttgtgtaaagtggaccaagagaaaaccatattgtggaagacagtttttcacacattttagtatccgtgttgcacactgtgttagagattatgaaaaccattctagttcagtttatcacccaaagctatcacccaacactaactccttagtattctttcaaaggaagcaaatagtaagattacaatatagaatgagctaaaatatcaaggggaaagcttcatttcctctttattctctttatcgtccttcccattttaaccttttaactttgttttgatggtgccaagttgagtctgatgtacccattgttagctttggatagaatttaaagtatatcccagaattagctgggcatgttggtgcatgcctgtaatcccagctactcaggaggcaaggctggagaatcgcttgaacccgggaggcggaggttgcagtgagctgagattgcgccattgcactccatcctgggagacaagagcgaaactctgcctcaaaaaaaaaagaatttaaagtatatcctttgaggtcattttatgtaacaattagatataacctcctgtgtatctctctgttcctcttcaaagttattaaaattcagcttaggtcatggatttttaatatgagggccagctgtactggtgtcaggtaggtgttttgggtgtagataatttaggactggagggctgagttaagttttaggaggagaatgtggttgagaatattttgaaaggaatttatggagtttgtgaaataacttcgaagtcctcttcctttacaatatttgaattcatatttgtaccttctcaaaatagtgattcatttttcctagaattacaggagggagctcttttactaatgttgttttgtttgcaactttgatggcttataataggaagtattctagttgtaaagaaaactctttagagacttttgactggtcagtatactgaggtgtgagatttgattcatgatgaagaaagcctatagattgccaaaaaattaattctccaaaccacctttcactctcagaaaatgagaccacaaaggagtatgctataaatcaaatttgccaaccaattatgtagatattactcattctaggactaatgatgatggtaaagaagttgccagtgttatggcaatgaaaatttcagaaaggaggagttgatgatcttctagatgtatatgaacacctgtctatatctgcatgtatatgttttgacctgcagtggttgcaatgttgatatgtgttcaagattattcctgtctacaaaactgaaggcccatgttcaaattgttctttattgggtgtttttatggtcacgtggtaacaattttcttacctaacctacaaaaggttctcttgatgaacatttttatttatatttactaatcttttttaaaaaaagctttcatagcattatataatcagatgaagaaagcccagtagaataaaaaaaaaattcattagcctagcctatattatgttttctgtcaaaggaaaacaaattctcaaataggaattctaaaaatatttactaaagtaaaataactacttaaaatgttttattccagttggaaggaaggtacagggagaaatcgcaattatttaggggagaagtatatttattataagatggtgtcctcaaattagcctaccatggcacgtaggggcagcagctatattagatttactagaggtgctaagttagaacactaggcttttattgaggcaggttttaatattgatagatgctttttgtttggtttgtttcttctgggagagaatggaggacttaagtagaagtagctactgataacagactttctagtagcagtttccactccacggttacctttttagtttcatagtatcttttcacaaagtattacaaataagctagattctcccagtttgggaatgcaagtttgctacatttttagcctggcaatatttgtgtaggtattgccttattggaaattctggaaacctgatactgcaacctgcaatgtaggatgtttgtatggcatttaaaggtaatggtgatgtttattattctatactttgcattctgtgagagtaattttcactctgtcttaagtgtgagtaagcctcttctaaaaatcttgttcttgccaagaaatttataaatcacatacgaagacgtctgttgctaacagttaactttatgaggtaactatatccttctatttctctggactcatttttaaaaaatatgccgaatactgcatactgtttaaggtagtatataagtttatgagagaagtggagagctttcttccttgaaaagtcggtatttgttgagataccatttgcctcacagagaggtgttccccactcccatccccattgccagataataaatattttgagaaaagtgacctaaaacagctgaaatcttaggtgcatctgtctgcagacctccttaagcaggctgtatcttacaattcccttactgcactgggtaagtgttaacttagtttttgttgtttgctcttttgctttaaatattctccaaattaccatttatgcaacatggttagggttaatactgcatggtattcatttatcttgtttcatgaactttccagtactgtacaaggtcaacaaagtaatgcctgtggtatcctcatctctcacttttttactctgtgattttagcacagtaaggtactgcaaagaccttccttccaaatgttctccttgactttatttcttgggccaattcagtatcctcaacatcctaagattttgttgttttatcactgacctgtggttggcctgttttattctaatttccagaaaagtcaagtcccagtatttgcaatatcaaataactctaaaaccgatgtgtgattctaccttccttactatttttactgggcaaatgccctatttttttaattattattatttttaacttttgggacacacaaaaatcagcaattctcatgaagcgtttgttagtgtggcagacttgtctaattcctgaaactcattcatccccttgagccagccaatggggaggaataggataatgcaaacacatgttttgttttctcattttcaaataatttaccatgttaaaataaacttttctttgttttttatttgtagagtcagctaagtacccatatttaaatgccgtctttattatttttttgaggtctttgtttttgtctgtttttgttttgttttgttttgtaaataaggtaactgggcaatcaaacaccttttggggattctggctttagtattttatcagccattttaaaattaaatataaaaatcctttgtaagaaacttgcatcctaatttttctttattgcaattgaaagtgtaaataataagacaatgtaagtaagaccttcctaatgtctaatacaaactgggctccagcaagtggccctatttttattagggttttgaaggtttgtgtgtgtgtgtgcgtgcgtgtgtgtgtttttcttttttaaatgtatagtagagtggtgtctgtataagtgttacctgtagtggggttttgtccagcaagcctgaaatttatactttgaaataaaactactgggtttttaacattc

SEQ ID NO: 8 - protein, 1278 aa, Homo sapiens
MHQDQRFRMDSLEEPQKKVFKARKTMRVSDRQQLEAVYKVKEELLKTDVKLLNGNHENGDLDPTSPLENMDYIKDKEEVNGIEEICFDPEGSKAEWKETPCILSVNVKNKQDDDLNCEPLSPHNITPEPVSKLPAEPVSGDPAPGDLDAGDPASGVLASGDSTSGDPTSSEPSSSDAASGDATSGDAPSGDVSPGDATSGDATADDLSSGDPTSSDPIPGEPVPVEPISGDCAADDIASSEITSVDLASGAPASTDPASDDLASGDLSSSELASDDLATGELASDELTSESTFDRTFEPKSVPVCEPVPEIDNIEPSSNKDDDFLEKNGADEKLEQIQSKDSLDEKNKADNNIDANEETLETDDTTICSDRPPENEKKVEEDIITELALGEDAISSSMEIDQGEKNEDETSADLVETINENVIEDNKSENILENTDSMETDEIIPILEKLAPSEDELTCFSKTSLLPIDETNPDLEEKMESSFGSPSKQESSESLPKEAFLVLSDEEDISGEKDESEVISQNETCSPAEVESNEKDNKPEEEEQVIHEDDERPSEKNEFSRRKRSKSEDMDNVQSKRRRYMEEEYEAEFQVKITAKGDINQKLQKVIQWLLEEKLCALQCAVFDKTLAELKTRVEKIECNKRHKTVLTELQAKIARLTKRFEAAKEDLKKRHEHPPNPPVSPGKTVNDVNSNNNMSYRNAGTVRQMLESKRNVSESAPPSFQTPVNTVSSTNLVTPPAVVSSQPKLQTPVTSGSLTATSVLPAPNTATVVATTQVPSGNPQPTISLQPLPVILHVPVAVSSQPQLLQSHPGTLVTNQPSGNVEFISVQSPPTVSGLTKNPVSLPSLPNPTKPNNVPSVPSPSIQRNPTASAAPLGTTLAVQAVPTAHSIVQATRTSLPTVGPSGLYSPSTNRGPIQMKIPISAFSTSSAAEQNSNTTPRIENQTNKTIDASVSKKAADSTSQCGKATGSDSSGVIDLTMDDEESGASQDPKKLNHTPVSTMSSSQPVSRPLQPIQPAPPLQPSGVPTSGPSQTTIHLLPTAPTTVNVTHRPVTQVTTRLPVPRAPANHQVVYTTLPAPPAQAPLRGTVMQAPAVRQVNPQNSVTVRVPQTTTYVVNNGLTLGSTGPQLTVHHRPPQVHTEPPRPVHPAPLPEAPQPQRLPPEAASTSLPQKPHLKLARVQSQNGIVLSWSVLEVDRSCATVDSYHLYAYHEEPSATVPSQWKKIGEVKALPLPMACTLTQFVSGSKYYFAVRAKDIYGRFGPFCDPQSTDVISSTQSS

SEQ ID NO: 9 - DNA, 5718 bp, Homo sapiens
  CDS (470)..(3790)
ctcctgaggctgccagcagccagcagtgactgcccgccctatctgggacccaggatcgctctgtgagcaacttggagccagagaggagatcaacaaggaggaggagagagccggcccctcagccctgctgcccagcagcagcctgtgctcgccctgcccaacgcagacagccagacccagggcggcccctctggcggctctgctcctcccgaaggatgcttggggagtgaggcgaagctgggccgctcctctcccctacagcagcccccttcctccatccctctgttctcctgagccttcaggagcctgcaccagtcctgcctgtccttctactcagctgttacccactctgggaccagcagtctttctgataactgggagagggcagtaaggaggacttcctggagggggtgactgtccagagcctggaactgtgcccacaccagaagccatcagcagcaaggacaccatgcggcttccgggtgcgatgccagctctggccctcaaaggcgagctgctgttgctgtctctcctgttacttctggaaccacagatctctcagggcctggtcgtcacacccccggggccagagcttgtcctcaatgtctccagcaccttcgttctgacctgctcgggttcagctccggtggtgtgggaacggatgtcccaggagcccccacaggaaatggccaaggcccaggatggcaccttctccagcgtgctcacactgaccaacctcactgggctagacacgggagaatacttttgcacccacaatgactcccgtggactggagaccgatgagcggaaacggctctacatctttgtgccagatcccaccgtgggcttcctccctaatgatgccgaggaactattcatctttctcacggaaataactgagatcaccattccatgccgagtaacagacccacagctggtggtgacactgcacgagaagaaaggggacgttgcactgcctgtcccctatgatcaccaacgtggcttttctggtatctttgaggacagaagctacatctgcaaaaccaccattggggacagggaggtggattctgatgcctactatgtctacagactccaggtgtcatccatcaacgtctctgtgaacgcagtgcagactgtggtccgccagggtgagaacatcaccctcatgtgcattgtgatcgggaatgaggtggtcaacttcgagtggacatacccccgcaaagaaagtgggcggctggtggagccggtgactgacttcctcttggatatgccttaccacatccgctccatcctgcacatccccagtgccgagttagaagactcggggacctacacctgcaatgtgacggagagtgtgaatgaccatcaggatgaaaaggccatcaacatcaccgtggttgagagcggctacgtgcggctcctgggagaggtgggcacactacaatttgctgagctgcatcggagccggacactgcaggtagtgttcgaggcctacccaccgcccactgtcctgtggttcaaagacaaccgcaccctgggcgactccagcgctggcgaaatcgccctgtccacgcgcaacgtgtcggagacccggtatgtgtcagagctgacactggttcgcgtgaaggtggcagaggctggccactacaccatgcgggccttccatgaggatgctgaggtccagctctccttccagctacagatcaatgtccctgtccgagtgctggagctaagtgagagccaccctgacagtggggaacagacagtccgctgtcgtggccggggcatgccccagccgaacatcatctggtctgcctgcagagacctcaaaaggtgtccacgtgagctgccgcccacgctgctggggaacagttccgaagaggagagccagctggagactaacgtgacgtactgggaggaggagcaggagtttgaggtggtgagcacactgcgtctgcagcacgtggatcggccactgtcggtgcgctgcacgctgcgcaacgctgtgggccaggacacgcaggaggtcatcgtggtgccacactccttgccctttaaggtggtggtgatctcagccatcctggccctggtggtgctcaccatcatctcccttatcatcctcatcatgctttggcagaagaagccacgttacgagatccgatggaaggtgattgagtctgtgagctctgacggccatgagtacatctacgtggaccccatgcagctgccctatgactccacgtgggagctgccgcgggaccagcttgtgctgggacgcaccctcggctctggggcctttgggcaggtggtggaggccacggctcatggcctgagccattctcaggccacgatgaaagtggccgtcaagatgcttaaatccacagcccgcagcagtgagaagcaagcccttatgtcggagctgaagatcatgagtcaccttgggccccacctgaacgtggtcaacctgttgggggcctgcaccaaaggaggacccatctatatcatcactgagtactgccgctacggagacctggtggactacctgcaccgcaacaaacacaccttcctgcagcaccactccgacaagcgccgcccgcccagcgcggagctctacagcaatgctctgcccgttgggctccccctgcccagccatgtgtccttgaccggggagagcgacggtggctacatggacatgagcaaggacgagtcggtggactatgtgcccatgctggacatgaaaggagacgtcaaatatgcagacatcgagtcctccaactacatggccccttacgataactacgttccctctgcccctgagaggacctgccgagcaactttgatcaacgagtctccagtgctaagctacatggacctcgtgggcttcagctaccaggtggccaatggcatggagtttctggcctccaagaactgcgtccacagagacctggcggctaggaacgtgctcatctgtgaaggcaagctggtcaagatctgtgactttggcctggctcgagacatcatgcgggactcgaattacatctccaaaggcagcacctttttgcctttaaagtggatggctccggagagcatcttcaacagcctctacaccaccctgagcgacgtgtggtccttcgggatcctgctctgggagatcttcaccttgggtggcaccccttacccagagctgcccatgaacgagcagttctacaatgccatcaaacggggttaccgcatggcccagcctgcccatgcctccgacgagatctatgagatcatgcagaagtgctgggaagagaagtttgagattcggccccccttctcccagctggtgctgcttctcgagagactgttgggcgaaggttacaaaaagaagtaccagcaggtggatgaggagtttctgaggagtgaccacccagccatccttcggtcccaggcccgcttgcctgggttccatggcctccgatctcccctggacaccagctccgtcctctatactgccgtgcagcccaatgagggtgacaacgactatatcatccccctgcctgaccccaaacccgaggttgctgacgagggcccactggagggttcccccagcctagccagctccaccctgaatgaagtcaacacctcctcaaccatctcctgtgacagccccctggagccccaggacgaaccagagccagagccccagcttgagctccaggtggagccggagccagagctggaacagttgccggattcggggtgccctgcgcctcgggcggaagcagaggatagcttcctgtagggggctggcccctaccctgccctgcctgaagctccccccctgccagcacccagcatctcctggcctggcctgaccgggcttcctgtcagccaggctgcccttatcagctgtccccttctggaagctttctgctcctgacgtgttgtgccccaaaccctggggctggcttaggaggcaagaaaactgcaggggccgtgaccagccctctgcctccagggaggccaactgactctgagccagggttcccccagggaactcagttttcccatatgtaagatgggaaagttaggcttgatgacccagaatctaggattctctccctggctgacaggtggggagaccgaatccctccctgggaagattcttggagttactgaggtggtaaattaacttttttctgttcagccagctacccctcaaggaatcatagctctctcctcgcacttttatccacccaggagctagggaagagaccctagcctccctggctgctggctgagctagggcctagccttgagcagtgttgcctcatccagaagaaagccagtctcctccctatgatgccagtccctgcgttccctggcccgagctggtctggggccattaggcagcctaattaatgctggaggctgagccaagtacaggacacccccagcctgcagcccttgcccagggcacttggagcacacgcagccatagcaagtgcctgtgtccctgtccttcaggcccatcagtcctggggctttttctttatcaccctcagtcttaatccatccaccagagtctagaaggccagacgggccccgcatctgtgatgagaatgtaaatgtgccagtgtggagtggccacgtgtgtgtgccagtatatggccctggctctgcattggacctgctatgaggctttggaggaatccctcaccctctctgggcctcagtttccccttcaaaaaatgaataagtcggacttattaactctgagtgccttgccagcactaacattctagagtattccaggtggttgcacatttgtccagatgaagcaaggccatataccctaaacttccatcctgggggtcagctgggctcctgggagattccagatcacacatcacactctggggactcaggaaccatgccccttccccaggcccccagcaagtctcaagaacacagctgcacaggccttgacttagagtgacagccggtgtcctggaaagcccccagcagctgccccagggacatgggaagaccacgggacctctttcactacccacgatgacctccgggggtatcctgggcaaaagggacaaagagggcaaatgagatcacctcctgcagcccaccactccagcacctgtgccgaggtctgcgtcgaagacagaatggacagtgaggacagttatgtcttgtaaaagacaagaagcttcagatgggtaccccaagaaggatgtgagaggtgggcgctttggaggtttgcccctcacccaccagctgccccatccctgaggcagcgctccatgggggtatggttttgtcactgcccagacctagcagtgacatctcattgtccccagcccagtgggcattggaggtgccaggggagtcagggttgtagccaagacgcccccgcacggggagggttgggaagggggtgcaggaagctcaacccctctgggcaccaaccctgcattgcaggttggcaccttacttccctgggatccccagagttggtccaaggagggagagtgggttctcaatacggtaccaaagatataatcacctaggtttacaaatatttttaggactcacgttaactcacatttatacagcagaaatgctattttgtatgctgttaagtttttctatctgtgtacttttttttaagggaaagattttaatattaaacctggtgcttctcactcacaaaaa

SEQ ID NO: 10 - protein, 1106 aa, Homo sapiens
MRLPGAMPALALKGELLLLSLLLLLEPQISQGLVVTPPGPELVLNVSSTFVLTCSGSAPVVWERMSQEPPQEMAKAQDGTFSSVLTLTNLTGLDTGEYFCTHNDSRGLETDERKRLYIFVPDPTVGFLPNDAEELFIFLTEITEITIPCRVTDPQLVVTLHEKKGDVALPVPYDHQRGFSGIFEDRSYICKTTIGDREVDSDAYYVYRLQVSSINVSVNAVQTVVRQGENITLMCIVIGNEVVNFEWTYPRKESGRLVEPVTDFLLDMPYHIRSILHIPSAELEDSGTYTCNVTESVNDHQDEKAINITVVESGYVRLLGEVGTLQFAELHRSRTLQVVFEAYPPPTVLWFKDNRTLGDSSAGEIALSTRNVSETRYVSELTLVRVKVAEAGHYTMRAFHEDAEVQLSFQLQINVPVRVLELSESHPDSGEQTVRCRGRGMPQPNIIWSACRDLKRCPRELPPTLLGNSSEEESQLETNVTYWEEEQEFEVVSTLRLQHVDRPLSVRCTLRNAVGQDTQEVIVVPHSLPFKVVVISAILALVVLTIISLIILIMLWQKKPRYEIRWKVIESVSSDGHEYIYVDPMQLPYDSTWELPRDQLVLGRTLGSGAFGQVVEATAHGLSHSQATMKVAVKMLKSTARSSEKQALMSELKIMSHLGPHLNVVNLLGACTKGGPIYIITEYCRYGDLVDYLHRNKHTFLQHHSDKRRPPSAELYSNALPVGLPLPSHVSLTGESDGGYMDMSKDESVDYVPMLDMKGDVKYADIESSNYMAPYDNYVPSAPERTCRATLINESPVLSYMDLVGFSYQVANGMEFLASKNCVHRDLAARNVLICEGKLVKICDFGLARDIMRDSNYISKGSTFLPLKWMAPESIFNSLYTTLSDVWSFGILLWEIFTLGGTPYPELPMNEQFYNAIKRGYRMAQPAHASDEIYEIMQKCWEEKFEIRPPFSQLVLLLERLLGEGYKKKYQQVDEEFLRSDHPAILRSQARLPGFHGLRSPLDTSSVLYTAVQPNEGDNDYIIPLPDPKPEVADEGPLEGSPSLASSTLNEVNTSSTISCDSPLEPQDEPEPEPQLELQVEPEPELEQLPDSGCPAPRAEAEDSFL

SEQ ID NO: 11 - DNA, 22 bp, Artificial Sequence
  Forward primer
caagtggaccatctcagaccac

SEQ ID NO: 12 - DNA, 22 bp, Artificial Sequence
  Reverse primer
atttaagcatcttgacggccac

SEQ ID NO: 13 - DNA, 30 bp, Artificial Sequence
  ATF7IP-1 Fwd
ttcagaatggacagtttagaagaacctcag

SEQ ID NO: 14 - DNA, 20 bp, Artificial Sequence
  ATF7IP-1 Rev
tctgctggagagcacgtttc

SEQ ID NO: 15 - DNA, 23 bp, Artificial Sequence
  ATF7IP-2 Fwd
aagcttgcaccttctgaggatga

SEQ ID NO: 16 - DNA, 20 bp, Artificial Sequence
  ATF7IP-2 Rev
atcggatctcgtaacgtggc

SEQ ID NO: 17 - DNA, 28 bp, Artificial Sequence
  ATF7IP-PDGFRB-3 Fwd
tggtcctctctgatgaagaggatatttc

SEQ ID NO: 18 - DNA, 20 bp, Artificial Sequence
  ATF7IP-PDGFRB-3 Rev
tcatcgtggcctgagaatgg

SEQ ID NO: 19 - DNA, 25 bp, Artificial Sequence
  EBF1-PDGFRB Fwd
atgtttgggattcaggaaagcatcc

SEQ ID NO: 20 - DNA, 20 bp, Artificial Sequence
  EBF1-PDGFRB Rev
atgcggtaaccccgtttgat

SEQ ID NO: 21 - DNA, 22 bp, Artificial Sequence
  ATF7IP-PDGFRB detection Fwd-1
cgtgaatgtaacacatcgtcca

SEQ ID NO: 22 - DNA, 21 bp, Artificial Sequence
  ATF7IP-PDGFRB detection Rev-1
agctcccacgtggagtcatag

SEQ ID NO: 23 - DNA, 20 bp, Artificial Sequence
  EBF1-PDGFRBdetection Fwd-1
gggcgtgaattcgttcagtg

SEQ ID NO: 24 - DNA, 20 bp, Artificial Sequence
  EBF1-PDGFRB detection Rev-1
atcggatctcgtaacgtggc

SEQ ID NO: 25 - DNA, 20 bp, Artificial Sequence
  WT-PDGFRB detection Fwd-1
tccagcaccttcgttctgac

SEQ ID NO: 26 - DNA, 20 bp, Artificial Sequence
  WT-PDGFRB detection Rev-1
ccagaaaagccacgttggtg

(19) United States
(12) Patent Application Publication
(10) Pub. No.: US 2016/0289663 A1
(43) Pub. Date: Oct. 6, 2016

(54) NOVEL CHIMERIC GENE ATF7IP-PDGFRB OF ACUTE LYMPHOBLASTIC LEUKEMIA

(71) Applicants: NATIONAL CENTER FOR CHILD HEALTH AND DEVELOPMENT, Tokyo (JP); NATIONAL CANCER CENTER, Tokyo (JP); LSIP, LLC, Tokyo (JP)

(72) Inventors: Nobutaka Kiyokawa, Tokyo (JP); Kenji Matsumoto, Tokyo (JP); Kenichiro Kobayashi, Tokyo (JP); Hitoshi Ichikawa, Tokyo (JP)

(21) Appl. No.: 15/037,907

(22) PCT Filed: Nov. 17, 2014
(86) PCT No.: PCT/JP2014/080306

§ 371 Date: May 19, 2016

(30) Foreign Application Priority Data
Nov. 22, 2013 (JP) 2013-242285

(51) Int. Cl. C12N 9/96; C12N 9/12; G01N 33/574; A61K 31/506; C07K 14/47

(52) U.S. Cl. CPC C12N 9/96; A61K 31/506; C07K 14/4702; G01N 33/57484; C12N 9/12; C12Y 207/10001; C07K 2319/00; G01N 2500/10

(57) ABSTRACT

To identify a mutation that can serve as an indicator for predicting the effectiveness of drug treatment in cancers such as leukemia; to provide a means for detecting said mutation; and to provide a means for identifying, based on said mutation, patients with cancer or subjects with a risk of cancer, in whom a drug targeting a gene having said mutation or a protein encoded by said gene shows a therapeutic effect.

A method for detecting a gene fusion serving as a responsible mutation (driver mutation) for cancer, the method comprising the step of detecting an ATF7IP-PDGFRB fusion polynucleotide or a polypeptide encoded thereby, in an isolated sample from a subject.

### TECHNICAL FIELD

The present invention relates mainly to a method for detecting a gene fusion serving as a responsible mutation for cancer, and a method for identifying patients with cancer or subjects with a risk of cancer, in whom a substance suppressing the expression and/or activity of a polypeptide encoded by a fusion polynucleotide produced by said gene fusion show a therapeutic effect.

### BACKGROUND ART

Cancer is the first-ranked disease among causes of death in Japan. Cancer cells are known to become malignant due to genetic alterations. Identifying genetic alterations characteristic of cancer type is expected to contribute not only to the clarification of carcinogenic mechanisms but also to, for example, the diagnosis and prognosis prediction of cancers and the development and selection of a cancer therapy.

Chimeric genes (hereinafter also referred to as “fusion genes”) are formed by a mechanism in which two genes encoding proteins which inherently function as completely distinct molecules fuse together due to abnormalities such as chromosomal translocation, and as a consequence, those chimeric genes produce fusion proteins having abnormal functions. Chimeric genes are known to trigger the development of leukemia and other cancers, and are used as indicators for diagnosis and as targets for drug discovery. Particularly in recent years, there have been increasing examples of successful development of molecular targeted therapies against cancers having chimeric genes associated with constituents (e.g., kinases) of carcinogenesis-related signaling pathways. For example, it is reported that tyrosine kinase inhibitors (e.g., crizotinib) targeting ALK protein are effective in the treatment of breast cancer patients bearing the EML4-ALK fusion gene, and that tyrosine kinase inhibitors (e.g., imatinib) targeting the protein encoded by the BCR-ABL fusion gene are effective in the treatment of leukemia patients bearing this fusion gene.

Acute lymphoblastic leukemia (ALL) is a cancer in which lymphocytes become malignant at an immature stage and grow abnormally. ALL develops at any age from infancy to adulthood, and is known as the most common cancer in infancy. As for ALL, many chimeric genes have been reported as genetic alterations. For example, Non Patent Literature 1 discloses that, in order to identify genetic alternations in IKZF1-mutated, BCR-ABL1-negative ALL with poor outcome (Ph-like ALL), RNA sequencing and whole genome sequencing were performed on B-progenitor ALL (B-ALL) cases identified as Ph-like ALL, thereby identifying the in-frame fusions NUP214-ABL1, EBF1-PDGFRB, BCR-JAK2, STRN3-JAK2, PAX5-JAK2, ETV6-ABL1, RANBP2-ABL1, and RCSD1-ABL1. This literature also discloses that the EBF1-PDGFRB, BCR-JAK2, and NUP214-ABL1 fusions are potential targets for tyrosine kinase inhibitor therapy.

### CITATION LIST

### Non Patent Literature

[Non Patent Literature 1] Roberts K. G., et al., Cancer Cell, 2012, 22 (2), 153-66.

### SUMMARY OF INVENTION

### Technical Problem

Identification of mutations in various cancers including ALL and other leukemias has not yet been made thoroughly, and there is still a demand for further identification of mutations that can serve as indicators for predicting the effectiveness of drug treatments.

Accordingly, the objects of the present invention include but are not limited to the following: to identify a mutation that can serve as an indicator for predicting the effectiveness of drug treatments in various cancers including ALL and other leukemias; to provide a means for detecting said mutation; and to provide a means for identifying, based on said mutations, patients with cancer or subjects with a risk of cancer, in whom a drug targeting a gene having said mutation or a protein encoded by said gene shows a therapeutic effect.

### Solution to Problem

As the result of conducting intensive studies with a view to achieving the above-mentioned objects, the present inventors identified the ATF7IP-PDGFRB gene as a novel fusion gene expressed in acute lymphoblastic leukemia (ALL). As for the PDGFRB gene, the fusion gene of this gene with the EBF1 gene has been reported in Non Patent Literature 1 noted above, but the presence of the fusion gene of this gene with the ATF7IP gene was wholly unexpected even considering any prior art literatures including the aforementioned one.

The ATF7IP-PDGFRB gene is considered to trigger the development of ALL as a result of producing a fusion protein between a SETDB1-binding protein (ATF7IP) and a tyrosine kinase (PDGFRB) and delivering a normally absent growth stimulus to cells. Therefore, targeted drugs such as tyrosine kinase inhibitors are highly likely to exhibit therapeutic effects against cancers positive for this gene fusion. In fact, it was confirmed that the tyrosine kinase inhibitor, dasatinib, is effective for ATF7IP-PDGFRB gene fusion-positive ALL patients.

The present inventors have made further studies based on these findings and completed the present invention.

More specifically, the present invention is defined as follows.

[1] A method for detecting a gene fusion, the method comprising the step of detecting an ATF7IP-PDGFRB fusion polypeptide comprising the SETDB1-binding domain of ATF7IP and the transmembrane region and kinase domain of PDGFRB, and having kinase activity, or a fusion polynucleotide encoding said polypeptide, in an isolated sample from a subject.
[2] The method according to [1], wherein the fusion polypeptide is any one of (i) to (iii) mentioned below:
(i) a polypeptide consisting of the amino acid sequence of SEQ ID NO: 2 or 4,
(ii) a polypeptide consisting of an amino acid sequence derived from the amino acid sequence of SEQ ID NO: 2 or 4 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2 or 4, and the polypeptide having kinase activity.
[3] The method according to [1] or [2], wherein the fusion polynucleotide is any one of (i) to (iv) mentioned below:
(i) a polynucleotide consisting of the nucleotide sequence of SEQ ID NO: 1 or 3,
(ii) a polynucleotide that hybridizes under stringent conditions with a polynucleotide consisting of a nucleotide sequence complementary to the polynucleotide consisting of the nucleotide sequence of SEQ ID NO: 1 or 3, and which encodes a polypeptide having kinase activity,
(iii) a polynucleotide that consists of a nucleotide sequence derived from the nucleotide sequence of SEQ ID NO: 1 or 3 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1 or 3, and which encodes a polypeptide having kinase activity.
[4] The method according to any one of [1] to [3], wherein the gene fusion is a responsible mutation (driver mutation) for cancer.
[5] The method according to [4], wherein the cancer is acute lymphoblastic leukemia.
[6] A method for identifying a patient with cancer or a subject with a risk of cancer, in whom a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting an ATF7IP-PDGFRB fusion polypeptide comprising the SETDB1-binding domain of ATF7IP and the transmembrane region and kinase domain of PDGFRB, and having kinase activity, or a fusion polynucleotide encoding said polypeptide, in an isolated sample from a subject, and
(2) determining that the substance suppressing the expression and/or activity of the polypeptide shows a therapeutic effect in the subject, in the case where the fusion polypeptide or the fusion polynucleotide encoding said polypeptide is detected.
[7] A kit for detecting a gene fusion, the kit comprising any or a combination of (A) to (C) mentioned below:
(A) a polynucleotide that serves as a probe designed to specifically recognize an ATF7IP-PDGFRB fusion polynucleotide;
(B) polynucleotides that serve as a pair of primers designed to enable specific amplification of an ATF7IP-PDGFRB fusion polynucleotide; or
(C) an antibody that specifically recognizes an ATF7IP-PDGFRB fusion polypeptide.
[8] The kit according to [7], wherein the gene fusion is a responsible mutation (driver mutation) for cancer.
[9] An isolated ATF7IP-PDGFRB fusion polypeptide or a fragment thereof, which comprises the SETDB1-binding domain of ATF7IP and the transmembrane region and kinase domain of PDGFRB, and has kinase activity.
[10] A polynucleotide encoding the fusion polypeptide or the fragment thereof according to [9].
[11] A therapeutic agent for ATF7IP-PDGFRB gene fusion-positive cancer.
[12] The therapeutic agent according to [11], comprising, as an active ingredient, a substance suppressing the expression and/or activity of an ATF7IP-PDGFRB fusion polypeptide comprising the SETDB1-binding domain of ATF7IP and the transmembrane region and kinase domain of PDGFRB, and having kinase activity.
[13] The therapeutic agent according to [11] or [12], wherein the cancer is acute lymphoblastic leukemia.
[14] The therapeutic agent according to any one of [11] to [13], wherein the substance suppressing the expression and/or activity of an ATF7IP-PDGFRB fusion polypeptide comprising the SETDB1-binding domain of ATF7IP and the transmembrane region and kinase domain of PDGFRB, and having kinase activity is a substance inhibiting the kinase activity of PDGFRB.
[15] The therapeutic agent according to [14], wherein the substance inhibiting the kinase activity of PDGFRB is imatinib mesylate, dasatinib, nilotinib, ponatinib, rebastinib or bafetinib.
[16] The therapeutic agent according to [14], wherein the substance inhibiting the kinase activity of PDGFRB is dasatinib.
[17] A drug for use in the treatment of cancer, the drug comprising a substance suppressing the expression and/or activity of an ATF7IP-PDGFRB fusion polypeptide comprising the SETDB1-binding domain of ATF7IP and the transmembrane region and kinase domain of PDGFRB, and having kinase activity.
[18] The drug according to [17], wherein the cancer is ATF7IP-PDGFRB gene fusion-positive cancer.
[19] The drug according to [17] or [18], wherein the cancer is acute lymphoblastic leukemia.
[20] The drug according to any one of [17] to [19], wherein the substance suppressing the expression and/or activity of an ATF7IP-PDGFRB fusion polypeptide comprising the SETDB1-binding domain of ATF7IP and the transmembrane region and kinase domain of PDGFRB, and having kinase activity is a substance inhibiting the kinase activity of PDGFRB, preferably imatinib mesylate, dasatinib, nilotinib, ponatinib, rebastinib or bafetinib, more preferably dasatinib.
[21] A method for treatment of cancer, comprising the step of administering to a subject a therapeutically effective amount of a substance suppressing the expression and/or activity of an ATF7IP-PDGFRB fusion polypeptide comprising the SETDB1-binding domain of ATF7IP and the transmembrane region and kinase domain of PDGFRB, and having kinase activity.
[22] The method according to [21], wherein the subject is a patient with ATF7IP-PDGFRB gene fusion-positive cancer.
[23] The method according to [21], wherein the subject is a patient with acute lymphoblastic leukemia.
[24] The method according to [21], wherein the substance suppressing the expression and/or activity of an ATF7IP-PDGFRB fusion polypeptide comprising the SETDB1-binding domain of ATF7IP and the transmembrane region and kinase domain of PDGFRB, and having kinase activity is a substance inhibiting the kinase activity of PDGFRB.
[25] The method according to [24], wherein the substance inhibiting the kinase activity of PDGFRB is imatinib mesylate, dasatinib, nilotinib, ponatinib, rebastinib or bafetinib.
[26] The method according to [24], wherein the substance inhibiting the kinase activity of PDGFRB is dasatinib.
[27] A method for screening a cancer therapeutic agent, the method comprising the steps of:
(1) bringing a test substance into contact with a cell that expresses an ATF7IP-PDGFRB fusion polypeptide comprising the SETDB1-binding domain of ATF7IP and the transmembrane region and kinase domain of PDGFRB, and having kinase activity;
(2) determining whether the expression and/or activity of the fusion polypeptide is suppressed or not; and
(3) selecting the substance determined to suppress the expression and/or activity of the fusion polypeptide, as a cancer therapeutic agent.

### Advantageous Effects of Invention

The present invention makes it possible to detect an unknown responsible mutation for the particular cancer, which has been first identified according to the present invention; to identify, based on the presence of said responsible mutation, patients with said cancer or subjects with a risk of said cancer, in whom a cancer treatment takes effect; and to treat said patients.

### BRIEF DESCRIPTION OF DRAWINGS

FIG. 1 is a schematic diagram showing an example of the domain structure of the ATF7IP-PDGFRB fusion protein. The downward arrows indicate the breakpoints in the ATF7IP protein and PDGFRB protein, and the point of fusion in the ATF7IP-PDGFRB fusion protein. Abbreviations: SETDB1, SETDB1-binding domain; MBD1, MBD1-binding domain; Ig, Ig-like domain; TM, Transmembrane region; and PTK, Kinase domain.

FIG. 2 is an electrophoretograph showing the results of detecting the ATF7IP-PDGFRB gene fusion by RT-PCR with cDNA synthesized from leukemic cell-derived RNA being used as a template. The arrow indicates the amplification of a gene fragment which implies a gene fusion.

FIG. 3 shows the results of nucleotide sequencing an amplified gene fragment.

FIG. 4 shows the clinical course of a patient with ATF7IP/PDGFRB-positive ALL. (A) The therapeutic response was evaluated by fluorescence in situ hybridization (FISH), RT-PCR (Kobayashi K., et al., Br J Haematol, 2014, 165, 836-841), and genomic PCR. Chemotherapy regimens were as follows: block 1: standard-risk ALL therapy (Tokyo Children's Cancer Study Group's study L99-15), maintenance therapy was started at 16 months after the initial diagnosis; block 2: ALL re-induction therapy; and block 3: AML regimen (Tsukimoto I., et al., J Clin Oncol, 2009, 27, 4007-4013). Abbreviations: CBSCT, cord blood stem cell transplantation; TM, tyrosine kinase inhibitor; and FISH, fluorescence in situ hybridization. (B) Results of detecting MRD in 17 samples using RT-PCR and genomic PCR. The levels of detection by genomic PCR and RT-PCR are indicated on the x-axis and γ-axis, respectively. In 2 samples, no MRD was detected by either of the two methods; in 4 samples, MRD was detected only by genomic PCR; and in 11 samples, MRD was detected within the quantitative range by both of the two methods. For the 4 samples with discordant results, the genomic PCR products were verified by clonal sequencing to rule out possibly incidental false-positive results. The dotted lines represent the thresholds for the quantifiable range of MRD detection.

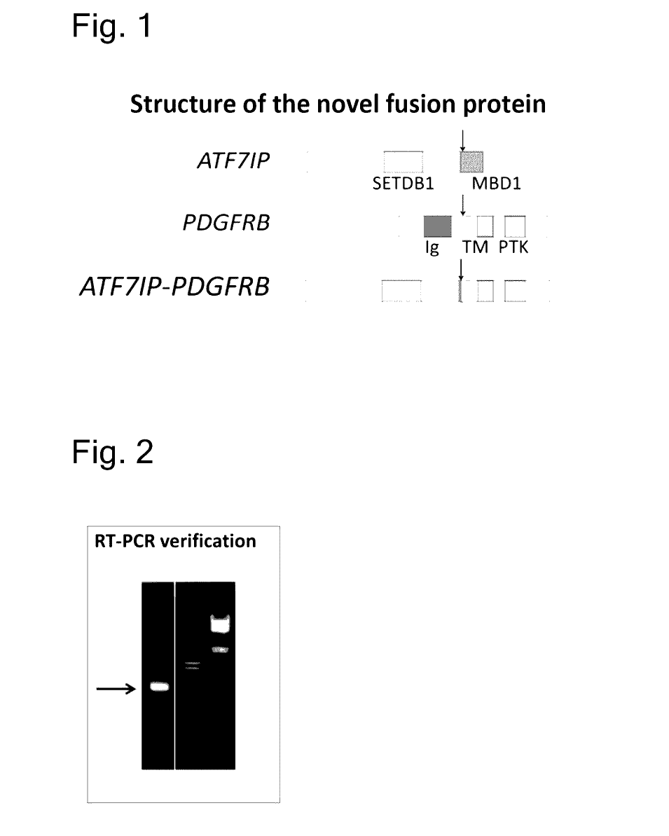

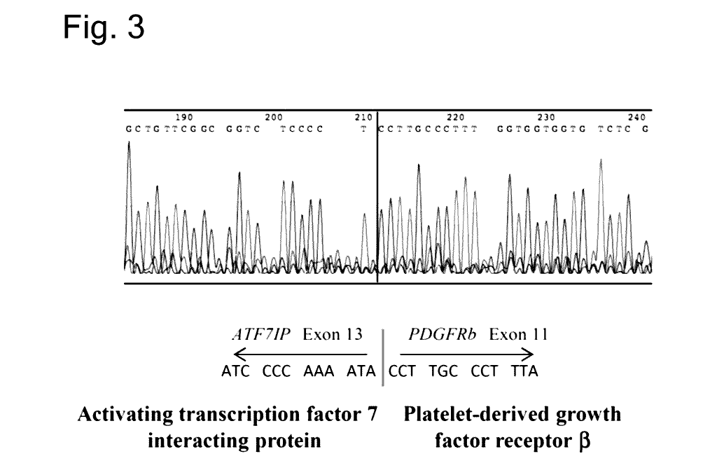

[FIG. 5-1] FIG. 5 illustrates that IL-3-independent cell proliferation is induced by ATF7IP- and EBF1-PDGFRB but not by wild-type (WT-) PDGFRB. (A) Ba/F3 cells were transfected with an ATF7IP-PDGFRB-expressing retroviral vector or an empty vector (Mock) by employing a tetracycline-inducible system. After 10-hour exposure to retrovirus followed by 24-hour treatment with or without doxycycline (Dox), the cells were washed to withdraw conditioned medium. After 60-hour incubation, the numbers of living cells were estimated by water-soluble tetrazolium salt (WST) assay. The mean±SEM of triplicates for each treatment is indicated by a bar graph.

[FIG. 5-2] (B) Cloned Ba/F3 cells transfected with ATF7IP-PDGFRB, EBF1-PDGFRB, and wild-type PDGFRB (WT-PDGFRB) were cultured in the absence of WEHI-3-conditioned medium (CM) for the indicated periods, and examined by WST assay. WT-PDGFRB transfectants were tested under the following two conditions, i.e., in the presence or absence of 100 ng/mL of PDGFBB. As a control, Ba/F3 cells transfected with an empty vector in the presence or absence of WEHI-3-conditioned medium were also examined. The OD values are expressed as the mean±SEM of triplicates. The data shown are representative of three independent experiments. (C) The results for WT-PDGFRB with or without PDGFBB and for Mock without conditioned medium, as shown in (B), were extracted and plotted on a graph with magnified Y-axis.

[FIG. 5-3] (D) The same sample preparations of WT-PDGFRB transfectants as in (B) were examined by Annexin V-binding assay. As controls, Mock cells treated with or without PDGFBB and/or conditioned medium were also similarly examined Experiments were performed in triplicate. For each type of samples, a typical cytogram and the mean±SEM of positivity (%) are presented. X-axis: fluorescence intensity; Y-axis: relative cell number.

FIG. 6 shows the expression of PDGFRB-related chimeric molecules and wild-type (WT-) PDGFRB in transfectants. (A) Total RNAs were extracted from 1×10⁷ Ba/F3 cells transfected with ATF7IP-PDGFRB (A), EBF1-PDGFRB (E), WT-PDGFRB (W), and an empty vector (Mock, M), and the expressions of the different genes and the internal control β-actin were examined by PCR. (B) The expressions of different types of PDGFRB were examined by immunoblot analysis. In the case of ATF7IP-PDGFRB, the fusion protein was detected by any of anti-ATF7IP antibody and anti-PDGFRB antibody. The gray arrows indicate several unexpected bands with smaller molecular weights than predicted, as detected in the case of WT-PDGFRB. (C) The expression of ATF7IP-PDGFRB in Ba/F3 cells was detected by immunocytochemistry.

FIG. 7 shows increased tyrosine phosphorylation mediated by the expression of PDGFRBs. (A) Cell lysates were prepared from Ba/F3 transfectants, and protein tyrosine phosphorylation was detected by immunoblotting using the anti-phosphotyrosine antibody 4G10. (B) The same sample preparations as in (A) were examined in the same way using phospho-specific anti-PDGFRB antibodies for the tyrosine residues indicated in the figure. Abbreviations: A, ATF7IP-PDGFRB; E, EBF1-PDGFRB; W, WT-PDGFRB; L, with ligand stimulation (100 ng/mL of PDGFBB); and M, Mock/empty vector.

FIG. 8 shows enhanced phosphorylation of signaling molecules located downstream of PDGFRB. The same sample preparations as in FIG. 7 were examined in the same way using the antibodies indicated in the figure. Abbreviations: A, ATF7IP-PDGFRB; E, EBF1-PDGFRB; W, WT-PDGFRB; +, with ligand stimulation (with 100 ng/mL of PDGFBB for 5 minutes); −, without ligand stimulation; and M, Mock/empty vector.

FIG. 9 shows phosphorylation and binding to PDGFRBs of c-Cbl in Ba/F3 transfectants. (A) Immunoprecipitation was performed on the indicated cell lysates using an anti-PDGFRB antibody. After separation on SDS-PAGE, immunoblot was performed with either an anti-c-Cbl or anti-PDGFRB antibody. (B) Using the same sample preparations, immunoprecipitation and immunoblot analysis were performed with anti-c-Cbl and phosphotyrosine antibodies, respectively. Abbreviations: A, ATF7IP-PDGFRB; E, EBF1-PDGFRB; W+, WT-PDGFRB with PDGFBB (100 ng/mL for 5 minutes); W-, WT-PDGFRB without PDGFBB; and M, Mock/empty vector.

FIG. 10 shows the effects of PI3 kinase and MEK inhibitors on proliferation of Ba/F3 transfectants bearing PDGFRBs. (A) ATF7IP-PDGFRB-expressing Ba/F3 cells in the absence of WEHI-3-conditioned medium (CM) and Mock Ba/F3 cells in the presence of CM were each treated with or without either the PI3 kinase inhibitor LY294002 or the MEK inhibitor PD98059 at the indicated concentrations for 24 hours. Subsequently, the number of living cells was estimated for each type of the cells by WST assay in the same way as in FIG. 5B. The ratio of the OD value obtained from the inhibitor-treated cells relative to that obtained from the inhibitor-untreated cells was calculated and expressed as the means±SEM of triplicates. (B) ATF7IP-PDGFRB-expressing Ba/F3 cells were treated with PD98059 in the presence or absence of CM, and examined in the same way as in (A).

FIG. 11 shows the effects of PI3 kinase and MEK inhibitors on the phosphorylation of AKT and MAP kinases. Cell lysates were prepared from the same sample preparations as in FIG. 10. Immunoblot analysis was performed in the same way as in FIG. 8. Abbreviations: None, without inhibitor; LY, with LY294002; and PD, with PD98059.

FIG. 12 shows the effects of PDGFBB and IL-3 on the phosphorylation of AKT and MAP kinases. WT-PDGFRB-expressing cells were maintained in the presence (CM(+)) or absence (CM(−)) of WEHI-3-conditioned medium for 12 hours. After stimulation with PDGFBB, cell lysates were prepared for the indicated periods. Immunoblot analysis was performed in the same way as in FIG. 8.

[FIG. 13-1] FIG. 13 shows the effects of tyrosine kinase inhibitors on proliferation of Ba/F3 transfectants bearing PDGFRBs. (A) ATF7IP- or EBF1-PDGFRB-expressing Ba/F3 cells and Mock Ba/F3 cells maintained in the presence of conditioned medium (CM(+)) were treated with or without tyrosine kinase inhibitors at the indicated concentrations for 24 hours. Subsequently, the number of living cells was estimated for each type of the cells by WST assay in the same way as in FIG. 5B. The ratio of the OD value obtained from the inhibitor-treated cells relative to that obtained from the inhibitor-untreated cells was calculated and expressed as the means±SEM of triplicates.

[FIG. 13-2] (B) The effects of nilotinib, bafetinib, rebastinib, and ponatinib on proliferation of Ba/F3 transfectants bearing PDGFRBs.

FIG. 14 shows the effects of tyrosine kinase inhibitors on phosphorylation of AKT and MAP kinases. Cell lysates were prepared from the same sample preparations treated with 100 nm of imatinib for 12 hours as in FIG. 13. Immunoblot analysis was performed as in FIG. 8.

### DESCRIPTION OF EMBODIMENTS

As disclosed below in Examples, the present inventors first found the ATF7IP-PDGFRB gene fusion serving as a responsible mutation (driver mutations) for cancer, in cancer tissues. On the basis of this finding, the present invention mainly provides a method for detecting said gene fusion; a method for identifying, based on the presence of said responsible mutation, a patient with cancer or a subject with a risk of cancer, in whom a cancer treatment takes effect; a method for treatment of cancer; a cancer therapeutic agent; and a method for screening a cancer therapeutic agent.

For the purpose of the present invention, the term “responsible mutation for cancer” is a term used interchangeably with the term “driver mutation”, and refers to a mutation that is present in cancer tissues and which is capable of inducing carcinogenesis of cells. Typically speaking, if a mutation is found in a cancer tissue in which none of known oncogene mutations exists (in other words, if a mutation exists mutually exclusively with the known oncogene mutations), then the mutation can be said as a responsible mutation for cancer.

<Specific Responsible Mutation for Cancer>

Hereafter, the inventive gene fusion is specifically explained. For the purpose of the present specification, the “point of fusion” in a fusion polynucleotide refers to a boundary that connects gene segments extending toward the 5′- and 3′-ends, in other words, a boundary between two nucleotide residues. The “point of fusion” in a fusion polypeptide refers to a boundary that connects polypeptides extending toward the N- and C-termini, in other words, a boundary between two amino acid residues, or, if a gene fusion occurs in one codon, one amino acid residue per se encoded by the codon.

The ATF7IP-PDGFRB gene fusion is a mutation that expresses a fusion protein between ATF7IP protein and PDGFRB protein (hereinafter also referred to as the “ATF7IP-PDGFRB fusion polypeptide”) and which is caused by a translocation (t(5;12)) having breakpoints in regions 12p13.1 and 5q33.1 of a human chromosome.

ATF7IP (activating transcription factor 7 interacting protein 1) is a protein also known as MCAF1, which is encoded by the gene located on chromosome 12p13.1 in humans, and is typified by a protein consisting of the amino acid sequence of SEQ ID NO: 6 or 8 (NCBI accession No. NP_060649.3 (26 Oct. 2013) or NP_851997.1 (8 Nov. 2013)). Although the function of ATF7IP protein is yet to be fully clarified, the protein is believed to bind a transcription factor to regulate its transcriptional activity. ATF7IP protein is characterized by having the SETDB1-binding domain and the MBD1-binding domain (FIG. 1). The SETDB1-binding domain corresponds to, for example, the amino acid sequence at positions 562 to 817 of the amino acid sequence of SEQ ID NO: 6 in humans. The MBD1-binding domain corresponds to, for example, the amino acid sequence at positions 1154 to 1270 of the amino acid sequence of SEQ ID NO: 6 in humans.

PDGFRB protein is a protein encoded by the gene located on chromosome 5q33.1 in humans, and is typified by a protein consisting of the amino acid sequence of SEQ ID NO: 10 (NCBI accession No. NP_002600.1 (27 Oct. 2013)). PDGFRB protein is characterized by having an Ig-like domain, a transmembrane region, and a kinase domain (FIG. 1). The Ig-like domain corresponds to, for example, the amino acid sequence at positions 228 to 415 of the amino acid sequence of SEQ ID NO: 10 in humans. The transmembrane region corresponds to, for example, the amino acid sequence at position 533 to 553 of the amino acid sequence of SEQ ID NO: 10 in humans. The kinase domain corresponds to, for example, the amino acid sequence at position 562 to 953 of the amino acid sequence of SEQ ID NO: 10 in humans.

The ATF7IP-PDGFRB fusion polypeptide is a polypeptide comprising the SETDB1-binding domain of ATF7IP protein and the transmembrane region and kinase domain of PDGFRB protein, and having kinase activity.

The ATF7IP-PDGFRB fusion polypeptide can contain all of the SETDB1-binding domain of ATF7IP protein, or can contain part of the SETDB1-binding domain as long as said part of the domain is capable of binding SETDB1. Whether said part of the SETDB1-binding domain is capable of binding SETDB1 can be confirmed by analysis by GST pull-down assay as described in J Biol Chem, 2005, 280 (14), 13928-35.

The ATF7IP-PDGFRB fusion polypeptide can contain all, part or none of the MBD1-binding domain of ATF7IP protein.

The ATF7IP-PDGFRB fusion polypeptide can contain all of the kinase domain of PDGFRB protein, or can contain part of the kinase domain as long as the ATF7IP-PDGFRB fusion polypeptide has kinase activity.

The expression saying that the ATF7IP-PDGFRB fusion polypeptide “has kinase activity” means that said fusion polypeptide is active as an enzyme phosphorylating tyrosine due to the kinase domain derived from PDGFRB protein. The kinase activity of the ATF7IP-PDGFRB fusion polypeptide is determined by a conventional method, and is commonly determined by incubating the fusion polypeptide with a substrate (e.g., synthetic peptide substrate) and ATP under appropriate conditions and then detecting phosphorylated tyrosine in the substrate. The kinase activity can also be measured using a commercially available measurement kit.

The ATF7IP-PDGFRB fusion polypeptide can contain all or part of the transmembrane region of PDGFRB protein, but is preferable to contain all of the transmembrane region.

In the present invention, the polynucleotide encoding the ATF7IP-PDGFRB fusion polypeptide (hereinafter also referred to as the “ATF7IP-PDGFRB fusion polynucleotide”) is a polynucleotide that encodes the polypeptide comprising the SETDB1-binding domain of ATF7IP protein and the transmembrane region and kinase domain of PDGFRB protein, and having kinase activity. The ATF7IP-PDGFRB fusion polynucleotide can be in the form of any of mRNA, cDNA and genomic DNA.

The ATF7IP-PDGFRB fusion polynucleotide according to the present invention can be, for example, an ATF7IP-PDGFRB fusion polynucleotide encoding the polypeptide mentioned below:

(i) a polypeptide consisting of the amino acid sequence of SEQ ID NO: 2 or 4,
(ii) a polypeptide consisting of an amino acid sequence derived from the amino acid sequence of SEQ ID NO: 2 or 4 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2 or 4, and the polypeptide having kinase activity.

As disclosed below in Examples, the amino acid sequence of SEQ ID NO: 2 or 4 is an amino acid sequence encoded by the ATF7IP-PDGFRB fusion polynucleotide comprising partial sequences, which was found in samples from human cancer tissues.

As used above in (ii), the phrase “one or more amino acids” refers to generally 1 to 50 amino acids, preferably 1 to 30 amino acids, more preferably 1 to 10 amino acids, still more preferably one to several amino acids (for example, 1 to 5 amino acids, 1 to 4 amino acids, 1 to 3 amino acids, 1 or 2 amino acids, or one amino acid).

As used above in (iii), the phrase “sequence identity of at least 80%” refers to a sequence identity of preferably at least 85%, more preferably at least 90% or at least 95%, still more preferably at least 97%, at least 98% or at least 99%. Amino acid sequence identity can be determined using the BLASTX or BLASTP program (Altschul S. F., et al., J. Mol. Biol., 1990, 215: 403) which is based on the BLAST algorithm developed by Karlin and Altschul (Proc. Natl. Acad. Sci. USA, 1990, 87: 2264-2268; and Proc. Natl. Acad. Sci. USA, 1993, 90: 5873). In the process of making amino acid sequence analysis using BLASTX, the parameter setting is typically made as follows: score=50 and wordlength=3. In the process of making amino acid sequence analysis using the BLAST and Gapped BLAST programs, the default parameters of these programs are used. The specific procedures for conducting these analyses are well known to those skilled in the art (e.g., http://www.ncbi.nlm.nih.gov/).

Also, the ATF7IP-PDGFRB fusion polynucleotide according to the present invention can be, for example, any one of the polynucleotides mentioned below:

(i) a polynucleotide consisting of the nucleotide sequence of SEQ ID NO: 1 or 3,
(ii) a polynucleotide that hybridizes under stringent conditions with a polynucleotide consisting of a nucleotide sequence complementary to the polynucleotide consisting of the nucleotide sequence of SEQ ID NO: 1 or 3, and which encodes a polypeptide having kinase activity,
(iii) a polynucleotide that consists of a nucleotide sequence derived from the nucleotide sequence of SEQ ID NO: 1 or 3 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1 or 3, and which encodes a polypeptide having kinase activity.

As disclosed below in Examples, the nucleotide sequence of SEQ ID NO: 1 or 3 is a nucleotide sequence of an ATF7IP-PDGFRB fusion polynucleotide comprising partial sequences, which is found in samples from human cancer tissues.

As used above in (ii), the phrase “under stringent conditions” refers to moderately or highly stringent conditions, unless particularly specified.

The moderately stringent conditions can be easily designed by those skilled in the art on the basis of, for example, the length of the polynucleotide of interest. Basic conditions are described in Sambrook, et al., Molecular Cloning: A Laboratory Manual, 3rd ed., ch. 6-7, Cold Spring Harbor Laboratory Press, 2001. Typically, the moderately stringent conditions comprise: prewashing of a nitrocellulose filter in 5×SSC, 0.5% SDS, 1.0 mM EDTA (pH 8.0); hybridization in ca. 50% formamide, 2-6×SSC at about 40-50° C. (or any other similar hybridization solution like a Stark's solution in ca. 50% formamide at about 42° C.); and washing of the filter in 0.5-6×SSC, 0.1% SDS at about 40° C.-60° C. The moderately stringent conditions preferably comprise hybridization in 6×SSC at about 50° C., and may comprise the prewashing and/or washing under the above-mentioned conditions.

The highly stringent conditions can also be easily designed by those skilled in the art on the basis of, for example, the length of the polynucleotide of interest. The highly stringent conditions involve a higher temperature and/or a lower salt concentration than the moderately stringent conditions. Typically, the highly stringent conditions comprise hybridization in 0.2-6×SSC, preferably 6×SSC, more preferably 2×SSC, still more preferably 0.2×SSC, at about 65° C. In any case, the highly stringent conditions preferably comprise washing in 0.2×SSC, 0.1% SDS at about 65-68° C.

In any case, as a buffer for use in hybridization, prewashing and washing, SSPE (1×SSPE: 0.15 M NaCl, 10 mM NaH₂PO₄, and 1.25 mM EDTA, pH 7.4) can be used in place of SSC (1×SSC: 0.15 M NaCl and 15 mM sodium citrate). In any case, washing can be done for about 15 minutes after the completion of hybridization.

As used above in (iii), the phrase “one or more nucleotides” refers to generally 1 to 50 nucleotides, preferably 1 to 30 nucleotides, more preferably 1 to 10 nucleotides, still more preferably one to several nucleotides (for example, 1 to 5 nucleotides, 1 to 4 nucleotides, 1 to 3 nucleotides, 1 or 2 nucleotides, or one nucleotide).

As used above in (iv), the phrase “sequence identity of at least 80%” refers to a sequence identity of preferably at least 85%, more preferably at least 90% or at least 95%, still more preferably at least 97%, at least 98% or at least 99%. Nucleotide sequence identity can be determined using the BLASTN program (Altschul S. F., et al., J. Mol. Biol., 1990, 215: 403) which is based on the above-mentioned BLAST algorithm. In the process of making nucleotide sequence analysis using BLASTN, the parameter setting is typically made as follows: score=100 and wordlength=12.

<Method for Detecting a Gene Fusion>

The present invention provides a method for detecting the ATF7IP-PDGFRB gene fusion (hereinafter also referred to as the “inventive detection method”). This gene fusion preferably causes a responsible mutation (driver mutation) for cancer. The inventive detection method comprises the step of detecting an ATF7IP-PDGFRB fusion polynucleotide or a polypeptide encoded thereby, in an isolated sample from a subject.

In the inventive detection method, the subject is not particularly limited as long as it is a mammal. Examples of the mammal include: rodents such as mouse, rat, hamster, chipmunk and guinea pig; rabbit, pig, cow, goat, horse, sheep, mink, dog, cat; and primates such as human, monkey, cynomolgus monkey, rhesus monkey, marmoset, orangutan, and chimpanzee, with human being preferred.

The subject may be not only a subject affected by cancer, but also a subject suspected of being affected by cancer or a subject with a future risk of cancer. The “cancer” to which the inventive detection method is to be applied is not particularly limited as long as it is a cancer in which the ATF7IP-PDGFRB gene fusion can be detected, with leukemia being preferred, acute lymphoblastic leukemia (ALL) being more preferred, and B-progenitor acute lymphoblastic leukemia (B-ALL) being particularly preferred.

The “isolated sample” from the subject encompasses not only biological samples (for example, cells, tissues, organs (e.g., small intestine, spleen, kidney, liver, stomach, lung, adrenal gland, heart, brain, pancreas, and aorta), body fluids (e.g., blood, lymphs, bone marrow fluid), digestive juices, sputum, bronchoalveolar/bronchial lavage fluids, urine, and feces), but also nucleic acid extracts from these biological samples (e.g., genomic DNA extracts, mRNA extracts, and cDNA and cRNA preparations from mRNA extracts) and protein extracts. The genomic DNA, mRNA, cDNA or protein can be prepared by those skilled in the art through considering various factors including the type and state of the sample and selecting a known technique suitable therefor. The sample may also be the one that is fixed with formalin or alcohol, frozen, or embedded in paraffin.

Further, the “isolated sample” is preferably derived from a tissue, organ or body fluid affected or suspected of being affected by the above-mentioned cancer, and more preferably derived from a blood or bone marrow fluid affected or suspected of being affected by the above-mentioned cancer. Examples of the “isolated sample” include, but are not limited to, cells derived from the blood or bone marrow fluid of a patient with leukemia (preferably ALL, more preferably B-ALL) or a subject suspected of being affected by leukemia, and Total RNAs extracted from said cells.

In the inventive detection method, the detection of a fusion polynucleotide or a polypeptide encoded thereby can be made using a per se known technique.

If the object to be detected is a transcript from a genomic DNA (mRNA, or cDNA prepared from mRNA), a fusion polynucleotide in the form of mRNA or cDNA can be detected using, for example, RT-PCR, sequencing, TaqMan probe method, Northern blotting, dot blotting, or cDNA microarray analysis.

If the object to be detected is a genomic DNA, a fusion polynucleotide in the form of genomic DNA can be detected using, for example, in situ hybridization (ISH), genomic PCR, sequencing, TaqMan probe method, Southern blotting, or genome microarray analysis.

Any of the above-mentioned detection techniques can be used alone or in combination. For example, since the ATF7IP-PDGFRB gene fusion is believed to contribute to carcinogenesis by expressing an ATF7IP-PDGFRB fusion polypeptide, it is also preferred that if a fusion polynucleotide in the form of genomic DNA is detected (e.g., by in situ hybridization or the like), production of a transcript or a protein should be further confirmed (e.g., by RT-PCR, immunostaining or the like).

If a fusion polynucleotide is detected by a hybridization technique (e.g., TaqMan probe method, Northern blotting, Southern blotting, dot blotting, microarray analysis, in situ hybridization (ISH)), there can be used a polynucleotide that serves as a probe designed to specifically recognize the fusion polynucleotide. As used herein, the phrase “specifically recognize the fusion polynucleotide” means that under stringent conditions, the probe distinguishes and recognizes the fusion polynucleotide from other polynucleotides, including wild-type genes from which to derive both segments of the fusion polynucleotide each extending from the point of fusion toward the 5′- or 3′-end.

Since biological samples (e.g., biopsy samples) obtained in the course of treatment or diagnosis are often fixed in formalin, it is preferred to use in situ hybridization in the inventive detection method, from the viewpoints that the genomic DNA to be detected is stable even when fixed in formalin and that the detection sensitivity is high.

According to in situ hybridization, the genomic DNA (fusion polynucleotide) encoding a fusion polypeptide can be detected by hybridizing, to such a biological sample, the following polynucleotide (a) or (b) which has a chain length of at least 15 nucleotides and serves as a probe(s) designed to specifically recognize said fusion polynucleotide:

(a) a polynucleotide serving as at least one probe selected from the group consisting of a probe that hybridizes to the nucleotide sequence of the fusion partner gene toward the 5′-end (ATF7IP gene) and a probe that hybridizes to the nucleotide sequence of the fusion partner gene toward the 3′-end (PDGFRB gene); or
(b) a polynucleotide serving as a probe that hybridizes to a nucleotide sequence containing the point of fusion between the fusion partner gene toward the 5′-end and the fusion partner gene toward the 3′-end.

The ATF7IP gene according to the present invention, as far as it is derived from humans, and is typified by a gene consisting of the DNA sequence from position 14518566 to position 14655864 in the genome sequence identified in Genbank accession No. NC_000012.11.

The PDGFRB gene according to the present invention, as far as it is derived from humans, and is typified by a gene consisting of the DNA sequence (encoded into complementary strand) from position 149493402 to position 149535447 in the genome sequence identified in Genbank accession No. NC_000005.9.

However, the DNA sequences of genes can change in nature (i.e., in a non-artificial way) due to their mutations and the like. Thus, such native mutants can also be encompassed by the present invention (the same applies hereinafter).

The polynucleotide mentioned in (a) according to the present invention can be of any type as far as it is capable of detecting the presence of the genomic DNA encoding a fusion polypeptide in the above-mentioned biological sample by hybridizing to a nucleotide sequence(s) targeted by said polynucleotide, i.e., the nucleotide sequence of the fusion partner gene toward the 5′-end (ATF7IP gene) and/or the nucleotide sequence of the fusion partner gene toward the 3′-end (PDGFRB gene); preferably, the polynucleotide (a) is any of the polynucleotides mentioned below in (a1) to (a3):

(a1) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”);
(a2) a combination of a polynucleotide that hybridizes to the nucleotide sequence of that region of the fusion partner gene toward the 5′-end which extends upstream from its breakpoint toward the 5′-end (this polynucleotide is hereinafter also referred to as the “5′ fusion partner gene probe 1”), and a polynucleotide that hybridizes to the nucleotide sequence of that region of the fusion partner gene toward the 5′-end which extends downstream from its breakpoint toward the 3′-end (this polynucleotide is hereinafter also referred to as the “5′ fusion partner gene probe 2”); and
(a3) a combination of a polynucleotide that hybridizes to the nucleotide sequence of that region of the fusion partner gene toward the 3′-end which extends upstream from its breakpoint toward the 5′-end (this polynucleotide is hereinafter also referred to as the “3′ fusion partner gene probe 2”), and a polynucleotide that hybridizes to the nucleotide sequence of that region of the fusion partner gene toward the 3′-end which extends downstream from its breakpoint toward the 3′-end (this polynucleotide is hereinafter also referred to as the “3′ fusion partner gene probe 1”).

As for (a1) to (a3) mentioned above, the nucleotide sequence of that region of the fusion partner gene toward the 5′-end which extends upstream from its breakpoint toward the 5′-end typically contains a cording region for all or part of the SETDB1-binding domain of ATF7IP protein.

As for (a1) to (a3) mentioned above, the nucleotide sequence of that region of the fusion partner gene toward the 3′-end which extends downstream from its breakpoint toward the 3′-end typically contains cording regions for all or part of the transmembrane region of PDGFRB protein and for all or part of the kinase domain of the same protein.

The polynucleotides mentioned above in (a1) can be exemplified by the polynucleotide combination mentioned below in (a1-1): (a1-1) a combination of a polynucleotide that hybridizes to a coding region for all or part of the SETDB1-binding domain of ATF7IP protein, and a polynucleotide that hybridizes to a cording region for all or part of the kinase domain of PDGFRB protein.

In the present invention, the region to which the polynucleotide for use for in situ hybridization as mentioned above in (a) is to hybridize (such a region is hereinafter referred to as the “target nucleotide sequence”) is preferred to be located not more than 1000000 nucleotides away from the point of fusion between the fusion partner gene toward the 5′-end (ATF7IP gene) and the fusion partner gene toward the 3′-end (PDGFRB gene), from the viewpoints of specificity for the target nucleotide sequence and detection sensitivity.

In the present invention, the polynucleotide for use for in situ hybridization as mentioned above in (b) can be of any type as far as it is capable of detecting the presence of the genomic DNA encoding a fusion polypeptide in the above-mentioned biological sample by hybridizing to a nucleotide sequence targeted by said polynucleotide, i.e., a nucleotide sequence containing the point of fusion between the fusion partner gene toward the 5′-end and the fusion partner gene toward the 3′-end; and typical examples of the polynucleotide (b) are those which each hybridize to a nucleotide sequence containing the point of fusion in the nucleotide sequence of SEQ ID NO: 1 or 3.

Further, in the present invention, the polynucleotide for use for in situ hybridization as mentioned above in (a) or (b) is preferred to be a group consisting of multiple types of polynucleotides which can cover the entire target nucleotide sequence, from the viewpoints of specificity for the target nucleotide sequence and detection sensitivity. In such a case, each of the polynucleotides constituting the group has a length of at least 15 nucleotides, preferably from 100 to 1000 nucleotides.

The polynucleotide for use for in situ hybridization as mentioned above in (a) or (b) is preferably labeled with a fluorescent dye or the like for the purpose of detection. Examples of such a fluorescent dye include, but are not limited to, DEAC, FITC, R6G, TexRed, and CyS. Aside from the fluorescent dye, the polynucleotide may also be labeled with a radioactive isotope (e.g., ¹²⁵I, ¹³¹I, ³H, ¹⁴C, ³³P, ³²P), an enzyme (e.g., β-galactosidase, β-glucosidase, alkaline phosphatase, peroxidase, malate dehydrogenase), or a luminescent substance (e.g., luminol, luminol derivative, luciferin, lucigenin, 3,3′-diaminobenzidine (DAB)).

When in situ hybridization is performed using a combination of 5′ fusion partner gene probe 1 and 3′ fusion partner gene probe 1, a combination of 5′ fusion partner gene probe 1 and 5′ fusion partner gene probe 2, or a combination of 3′ fusion partner gene probe 2 and 3′ fusion partner gene probe 1, the probes of each combination are preferably labeled with different dyes from each other. If, as the result of in situ hybridization using such a combination of probes labeled with different dyes, an overlap is observed between the signal (e.g., fluorescence) emitted from the label on 5′ fusion partner gene probe 1 and the signal emitted from the label on 3′ fusion partner gene probe 1, then it can be determined that a genomic DNA encoding a fusion polypeptide of interest has been detected successfully. Also, if a split is observed between the signal emitted from the label on 5′ fusion partner gene probe 1 and the signal emitted from the label on 5′ fusion partner gene probe 2, or between the signal emitted from the label on 3′ fusion partner gene probe 2 and the signal emitted from the label on 3′ fusion partner gene probe 1, then it can be determined that a genomic DNA encoding a fusion polypeptide of interest has been detected successfully.

Polynucleotide labeling can be effected by a known method. For example, polynucleotides can be labeled by nick translation or random priming, in which the polynucleotides are caused to incorporate substrate nucleotides labeled with a fluorescent dye or the like.

The conditions for hybridizing the polynucleotide mentioned above in (a) or (b) to the above-mentioned biological sample by in situ hybridization can vary with various factors including the length of said polynucleotide; and exemplary highly stringent hybridization conditions are 0.2×SSC at 65° C., and exemplary low stringent hybridization conditions are 2.0×SSC at 50° C. Those skilled in the art could realize comparable stringent hybridization conditions to those mentioned above, by appropriately selecting salt concentration (e.g., SSC dilution rate), temperature, and various other conditions including concentrations of surfactant (e.g., NP-40) and formamide, and pH.

Besides the in situ hybridization, other examples of the method for detecting a genomic DNA encoding a fusion polypeptide of interest using the polynucleotide mentioned above in (a) or (b) include Southern blotting, Northern blotting and dot blotting. According to these methods, the fusion gene of interest is detected by hybridizing said polynucleotide (a) or (b) to a membrane in which a nucleic acid extract from the above-mentioned biological sample is transcribed. In the case of using said polynucleotide (a), if a polynucleotide that hybridizes to the nucleotide sequence of the fusion partner gene toward the 5′-end and a polynucleotide that hybridizes to the nucleotide sequence of a fusion partner gene toward the 3′-end both recognize the same band developed in the membrane, then it can be determined that a genomic DNA encoding the fusion polypeptide of interest has been detected successfully.

Additional examples of the method for detecting a genomic DNA encoding a fusion polypeptide of interest using said polynucleotide (b) include genome microarray analysis and DNA microarray analysis. According to these methods, the genomic DNA is detected by preparing an array in which said polynucleotide (b) is immobilized on a substrate and bringing the above-mentioned biological sample into contact with the polynucleotide immobilized on the array. The substrate is not particularly limited as long as it allows conversion of an oligo- or polynucleotide into a solid phase, and examples include glass plate, nylon membrane, microbeads, silicon chip, and capillary.

In the inventive detection method, it is also preferred to detect a fusion polynucleotide of interest using PCR.

In the process of PCR, there can be used polynucleotides serving as a pair of primers designed to specifically amplify a fusion polynucleotide using DNA (e.g., genomic DNA, cDNA) or RNA prepared from the above-mentioned biological sample as a template. As used herein, the phrase “specifically amplify a fusion polynucleotide” means that the primers do not amplify wild-type genes from which to derive both segments of a fusion polynucleotide of interest each extending from the point of fusion toward the 5′- or 3′-end, but can amplify said fusion polynucleotide alone. It is acceptable to amplify all of the fusion polynucleotide or to amplify that part of the fusion polynucleotide which contains the point of fusion.

The “polynucleotides serving as a pair of primers” to be used for PCR or the like consist of a sense primer (forward primer) and an anti-sense primer (reverse primer) that specifically amplify a target fusion polynucleotide. The sense primer is designed from the nucleotide sequence of that region of said fusion polynucleotide which extends from the point of fusion toward the 5′-end. The anti-sense primer is designed from the nucleotide sequence of that region of said fusion polynucleotide which extends from the point of fusion toward the 3′-end. From the viewpoints of the accuracy and sensitivity of PCR detection, these primers are commonly designed such that a PCR product of not more than 5 kb in size can be amplified. The primers can be designed as appropriate by a known method, for example, using the Primer Express® software (Applied Biosystems). The length of these polynucleotides is generally not less than 15 nucleotides (preferably not less than 16, 17, 18, 19 or 20 nucleotides, more preferably not less than 21 nucleotides) and not more than 100 nucleotides (preferably not more than 90, 80, 70, 60, 50 or 40 nucleotides, more preferably not more than 30 nucleotides).

Preferred examples of the “polynucleotides serving as a pair of primers” include: a primer set that consists of a polynucleotide consisting of the nucleotide sequence of SEQ ID NO: 11 and a polynucleotide consisting of the nucleotide sequence of SEQ ID NO: 12; and a primer set that consists of a polynucleotide consisting of the nucleotide sequence of SEQ ID NO: 21 and a polynucleotide consisting of the nucleotide sequence of SEQ ID NO: 22.

In the process of detecting a fusion polynucleotide by PCR, sequencing is performed on PCR products to sequence a nucleotide sequence containing the point of fusion, whereby it can be confirmed that a gene segment toward the 5′-end and a gene segment toward the 3′-end are joined in-frame and/or that a specified domain is contained in the fusion polynucleotide. Sequencing can be done by a known method—it can be easily done by using a sequencer (e.g., ABI-PRISM 310 Genetic Analyzer (Applied Biosystems Inc.)) in accordance with its operating instructions.

Also, in the process of detecting a fusion polynucleotide by PCR, it can be confirmed by the TaqMan probe method that a gene segment toward the 5′-end and a gene segment toward the 3′-end are joined in-frame and/or that a specified domain is contained in the fusion polynucleotide. The probe to be used in the TaqMan probe method can be exemplified by the polynucleotide mentioned above in (a) or (b). The probe is labeled with a reporter dye (e.g., FAM, FITC, VIC) and a quencher (e.g., TAMRA, Eclipse, DABCYL, MGB).

The above-mentioned primers and probes can be in the form of DNA, RNA, or DNA/RNA chimera, and is preferably in the form of DNA. Alternatively, the primers and probes may be such that part or all of the nucleotides are substituted by an artificial nucleic acid such as PNA (polyamide nucleic acid: a peptide nucleic acid), LNA™ (Locked Nucleic Acid; a bridged nucleic acid), ENA® (2′-0,4′-C-Ethylene-bridged Nucleic Acid), GNA (glycerol nucleic acid) or TNA (threose nucleic acid). Further, the primers and probes can be double- or single-stranded, and are preferably single-stranded.

As far as the primers and probes are capable of specifically hybridizing to a target sequence, they may contain one or more nucleotide mismatches, generally have at least 80% identity, preferably at least 90%, at least 91%, at least 92%, at least 93%, or at least 94% identity, more preferably at least 95%, at least 96%, at least 97%, at least 98%, or at least 99% identity, and most preferably 100% identity, to a sequence complementary to the target sequence.

The primers and probes can be synthesized, for example, according to a conventional method using an automatic DNA/RNA synthesizer on the basis of the information on the nucleotide sequences disclosed in the present specification.

In the inventive detection method, it is also acceptable to detect a fusion polynucleotide of interest by whole-transcriptome sequencing (RNA sequencing) or genome sequencing. These techniques can be carried out, for example, using a next-generation sequencer (e.g., Genome Analyzer IIx (Illumina), HiSeq sequencer (HiSeq 2000, Illumina), Genome Sequencer FLX System (Roche)), or the like according to the manufacturer's instructions. RNA sequencing can be done by, for example, preparing a cDNA library from a total RNA using a commercially available kit (e.g., mRNA-Seq sample preparation kit (Illumina)) according to the manufacturer's instructions and sequencing the prepared library using a next-generation sequencer.

In the inventive detection method, if the object to be detected is a translation product of a fusion polynucleotide (i.e., fusion polypeptide), the translation product can be detected using, for example, immunostaining, Western blotting, RIA, ELISA, flow cytometry, immunoprecipitation, or antibody array analysis. These techniques use an antibody that specifically recognizes a fusion polypeptide. As used herein, the phrase “specifically recognizes a fusion polypeptide” means that the antibody does not recognize other proteins than said fusion polypeptide, including wild-type proteins from which to derive both segments of said fusion polynucleotide each extending from the point of fusion toward the N- or C-terminus, but recognizes said fusion polypeptide alone. The antibody that “specifically recognizes a fusion polypeptide”, which is to be used in the inventive detection method, can be one antibody or a combination of two or more antibodies.

The “antibody that specifically recognizes a fusion polypeptide” can be exemplified by an antibody specific to a polypeptide containing the point of fusion of said fusion polypeptide (hereinafter referred to as the “fusion point-specific antibody”). As referred to herein, the “fusion point-specific antibody” means an antibody that specifically binds to the polypeptide containing said point of fusion but does not bind to wild-type proteins from which to derive the segments of the fusion polypeptide each extending toward the N- or C-terminus.

Also, the “antibody that specifically recognizes a fusion polypeptide” can be exemplified by a combination of an antibody binding to a polypeptide consisting of that region of the fusion polypeptide which extends from the point of fusion toward the N-terminus and an antibody binding to a polypeptide consisting of that region of the fusion polypeptide which extends from the point of fusion toward the C-terminus. The fusion polypeptide can be detected by performing sandwich ELISA, immunostaining, immunoprecipitation, Western blotting or the like using these two antibodies.

In the present invention, examples of the antibodies include, but are not limited to, natural antibodies such as polyclonal antibodies and monoclonal antibodies (mAb), and chimeric, humanized and single-stranded antibodies which can be prepared using genetic recombination techniques, and binding fragments thereof. The “binding fragments” refers to partial regions of the above-mentioned antibodies which have specific binding activity, and specific examples include Fab, Fab′, F(ab′)₂, Fv, and single-chain antibodies. The class of antibody is not particularly limited, and any antibody having any isotype, such as IgG, IgM, IgA, IgD or IgE, is acceptable, with IgG being preferred in consideration of ease of purification or other factors.

The “antibody that specifically recognizes a fusion polypeptide” can be prepared by those skilled in the art through selection of a known technique as appropriate. Examples of such a known technique include: a method in which a polypeptide containing the point of fusion of the fusion polypeptide, a polypeptide consisting of that region of the fusion polypeptide which extends from the point of fusion toward the N-terminus, or a polypeptide consisting of that region of the fusion polypeptide which extends from the point of fusion toward the C-terminus is inoculated into an immune animal, the immune system of the animal is activated, and then the serum (polyclonal antibody) of the animal is collected; as well as monoclonal antibody preparation methods such as hybridoma method, recombinant DNA method, and phage display method. Commercially available antibodies may also be used. If an antibody having a labeling agent attached thereto is used, the target protein can be detected directly by detecting this label. The labeling agent is not particularly limited as long as it is capable of binding to an antibody and is detectable, and examples include peroxidase, β-D-galactosidase, microperoxidase, horseradish peroxidase (HRP), fluorescein isothiocyanate (FITC), rhodamine isothiocyanate (RITC), alkaline phosphatase, biotin, and radioactive materials. Besides the direct detection of the target protein using the antibody having a labeling agent attached thereto, the target protein can also be detected indirectly using a secondary antibody having a labeling agent attached thereto, Protein G or A, or the like.

<Kit for Detecting a Gene Fusion>

As described above, a fusion polynucleotide produced by the ATF7IP-PDGFRB gene fusion, or a polypeptide encoded thereby, can be detected using such a primer, probe, or antibody or a combination thereof as mentioned above, whereby the gene fusion can be detected. Thus, the present invention provides a kit for detecting a gene fusion (preferably a gene fusion serving as a responsible mutation (driver mutation) for cancer), the kit comprising any or a combination of the polynucleotides and antibody mentioned below (hereinafter also referred to as the “inventive kit”):

(A) a polynucleotide that serves as a probe designed to specifically recognize an ATF7IP-PDGFRB fusion polynucleotide;
(B) polynucleotides that serve as a pair of primers designed to enable specific amplification of an ATF7IP-PDGFRB fusion polynucleotide; or
(C) an antibody that specifically recognizes an ATF7IP-PDGFRB fusion polypeptide.

In addition to the above-mentioned polynucleotide(s) or antibody, the inventive kit can also contain an appropriate combination of other components, including: a substrate required for detecting a label attached to the polynucleotide(s) or the antibody; a positive control (e.g., ATF7IP-PDGFRB fusion polynucleotide, ATF7IP-PDGFRB fusion polypeptide, or cells bearing the same); a negative control; a PCR reagent; a counterstaining reagent for use for in situ hybridization or the like (e.g., DAPI); a molecule required for antibody detection (e.g., secondary antibody, Protein G, Protein A); and a buffer solution for use in sample dilution or washing. The inventive kit can also contain instructions for use thereof. The above-mentioned inventive detection method can be easily carried out by using the inventive kit.

The inventive detection method and kit, which enable detection of the gene fusion newly discovered as a responsible mutation for cancer, are very useful in identifying subjects positive for said gene fusion and applying personalized medicine to each of the subjects, as described below.

<Method for Identifying a Patient with Cancer or a Subject with a Risk of Cancer>

The ATF7IP-PDGFRB gene fusion, serving as a responsible mutation for cancer, is believed to lead to constitutive activation of PDGFRB kinase activity, thereby contributing to malignant transformation of cancers. Thus, cancer patients in whom said gene fusion is detected are highly probable to be responsive to the treatment with a substance suppressing the expression and/or activity of a polypeptide encoded by a fusion polynucleotide produced by said gene fusion.

Thus, the present invention provides a method for identifying a patient with cancer or a subject with a risk of cancer, in whom a substance suppressing the expression and/or activity of a polypeptide encoded by a fusion polynucleotide produced by a gene fusion serving as a responsible mutation (driver mutation) for cancer shows a therapeutic effect (hereinafter also referred to as the “inventive identification method”).

The inventive identification method comprises the steps of:

(1) detecting an ATF7IP-PDGFRB fusion polypeptide comprising the SETDB1-binding domain of ATF7IP and the transmembrane region and kinase domain of PDGFRB, and having kinase activity, or a fusion polynucleotide encoding said polypeptide, in an isolated sample from a subject, and
(2) determining that a substance suppressing the expression and/or activity of the polypeptide shows a therapeutic effect in the subject, in the case where the fusion polypeptide or the fusion polynucleotide encoding said polypeptide is detected.

In the inventive identification method, the phrase “a patient with cancer or a subject with a risk of cancer” refers to a mammal, preferably human, which is, or is suspected of being, affected by cancer. The “cancer” to which the inventive identification method is to be applied is not particularly limited as long as it is a cancer in which the ATF7IP-PDGFRB gene fusion can be detected, with leukemia being preferred, acute lymphoblastic leukemia (ALL) being more preferred, and B-progenitor acute lymphoblastic leukemia (B-ALL) being particularly preferred.

In the inventive identification method, the “therapeutic effect” is not particularly limited as long as it is a beneficial effect of cancer treatment for a patient, and examples include a tumor shrinkage effect, a progression-free survival prolongation effect, and a life lengthening effect.

In the inventive identification method, the “substance suppressing the expression and/or activity of a polypeptide encoded by a fusion polynucleotide produced by a gene fusion serving as a responsible mutation (driver mutation) for cancer”, which is to be evaluated for its effectiveness in the treatment of cancer associated with the ATF7IP-PDGFRB gene fusion (this substance is hereinafter also referred to as the “substance to be evaluated in the inventive identification method”), is not particularly limited as long as it is a substance that directly or indirectly inhibits the expression and/or function of an ATF7IP-PDGFRB fusion polypeptide.

Examples of the substance inhibiting the expression of an ATF7IP-PDGFRB fusion polypeptide include: siRNAs (small interfering RNAs), shRNAs (short hairpin RNA), miRNAs (micro RNAs), and antisense nucleic acids which suppress the expression of an ATF7IP-PDGFRB fusion polypeptide; expression vectors capable of expressing these polynucleotides; and low-molecular-weight compounds.

Examples of the substance inhibiting the function of an ATF7IP-PDGFRB fusion polypeptide include substances inhibiting PDGFRB kinase activity (e.g., low-molecular-weight compounds), and antibodies binding to an ATF7IP-PDGFRB fusion polypeptide.

These substances may be substances that specifically suppress the expression and/or activity of an ATF7IP-PDGFRB fusion polypeptide, or may be substances that suppress even the expression and/or activity of wild-type PDGFRB protein. Specific examples of such substances include substances inhibiting PDGFRB kinase activity (i.e., tyrosine kinase inhibitors), such as imatinib mesylate (GLEEVEC®), dasatinib, nilotinib, ponatinib, rebastinib and bafetinib, with dasatinib being preferred.

These substances can be prepared by a per se known technique on the basis of the sequence information of an ATF7IP-PDGFRB fusion polynucleotide and/or an ATF7IP-PDGFRB fusion polypeptide which are disclosed in the present specification, or other data. Commercially available substances may also be used.

These substances are effective as cancer therapeutic agents for a subject, in the case where an ATF7IP-PDGFRB fusion polynucleotide or a polypeptide encoded thereby is detected in an isolated sample from said subject.

Step (1) in the inventive identification method can be carried out in the same way as the step included in the inventive detection method mentioned above.

At step (2) in the inventive identification method, the substance to be evaluated in the inventive identification method is determined to show a therapeutic effect in a subject (i.e., a patient with cancer or a subject with a risk of cancer), in the case where a fusion polynucleotide of interest or a polypeptide encoded thereby is detected in an isolated sample from the subject at step (1); however, the substance to be evaluated in the inventive identification method is determined to be unlikely to show a therapeutic effect in the subject, in the case where neither the fusion polynucleotide of interest nor the polypeptide encoded thereby is detected.

According to the inventive identification method, it is possible to detect a subject positive for the gene fusion newly discovered as a responsible mutation for cancer from among patients with cancer or subjects with a risk of cancer, and to identify a patient with cancer or a subject with a risk of cancer, in whom a substance suppressing the expression and/or activity of a polypeptide encoded by a fusion polynucleotide produced by said gene fusion shows a therapeutic effect; thus, the present invention is useful in that it makes it possible to provide a suitable treatment for such a subject.

<Method for Treatment of Cancer and Cancer Therapeutic Agent>

As described above, the inventive identification method identifies a patient with cancer, in whom a substance suppressing the expression and/or activity of a polypeptide encoded by a fusion polynucleotide produced by the ATF7IP-PDGFRB gene fusion shows a therapeutic effect. Thus, efficient cancer treatment can be performed by administering said substance selectively to a cancer patient who carries said fusion gene. Therefore, the present invention provides a method for treating cancer, comprising the step of administering said substance to a subject in whom said substance is determined to show a therapeutic effect by the inventive identification method mentioned above (hereinafter also referred to as the “inventive treatment method”).

Also, since the substance to be administered in the inventive treatment method functions as a cancer therapeutic agent, the present invention further provides a cancer therapeutic agent comprising, as an active ingredient, a substance suppressing the expression and/or activity of a polypeptide encoded by a fusion polynucleotide produced by the ATF7IP-PDGFRB gene fusion (hereinafter also referred to as the “inventive cancer therapeutic agent”).

In the context of the inventive treatment method and inventive cancer therapeutic agent, the cancer is not particularly limited as long as it is ATF7IP-PDGFRB gene fusion-positive cancer, with leukemia being preferred, acute lymphoblastic leukemia (ALL) being more preferred, and B-progenitor acute lymphoblastic leukemia (B-ALL) being particularly preferred. As referred to herein, the “ATF7IP-PDGFRB gene fusion-positive cancer” refers to a cancer in which an ATF7IP-PDGFRB fusion polynucleotide or a polypeptide encoded thereby is detected by the inventive detection method.

The inventive cancer therapeutic agent can be exemplified by the substances that are mentioned, in relation to the inventive identification method, as examples of the substance suppressing the expression and/or activity of a polypeptide encoded by a fusion polynucleotide produced by the ATF7IP-PDGFRB gene fusion.

The inventive cancer therapeutic agent can be formulated into a pharmaceutical composition with the use of a pharmaceutically acceptable carrier, excipient and/or other additives which are commonly used in pharmaceutical manufacturing.

The method for administering the inventive cancer therapeutic agent is selected as appropriate depending on various factors including the type of this suppressing agent and the type of cancer, and exemplary modes of administration that can be adopted include oral, intravenous, intraperitoneal, transdermal, intramuscular, intratracheal (aerosol), rectal and intravaginal administrations

The dose of the inventive cancer therapeutic agent can be determined as appropriate in consideration of various factors, including the activity and type of an active ingredient, the mode of administration (e.g., oral or parenteral administration), the severity of a disease, and the animal species, drug receptivity, body weight, and age of the subject to be treated with the inventive agent.

The treatment method and cancer therapeutic agent of the present invention are useful in that they allow treatment of patients having the particular responsible mutation for cancer which has been conventionally unknown and was first discovered according to this invention.

<Method for Screening a Cancer Therapeutic Agent>

The present invention provides a method for screening a cancer therapeutic agent which shows a therapeutic effect in a cancer patient having the ATF7IP-PDGFRB gene fusion (hereinafter also referred to as the “inventive screening method”). According to the inventive screening method, a substance suppressing the expression and/or activity of an ATF7IP-PDGFRB fusion polypeptide can be obtained as a cancer therapeutic agent.

The test substance to be subjected to the inventive screening method can be any compound or composition, and can be exemplified by nucleic acids (e.g., nucleoside, oligonucleoside, polynucleoside), saccharides (e.g., monosaccharide, disaccharide, oligosaccharide, polysaccharide), fats (e.g., saturated or unsaturated, straight-chain, branched-chain and/or cyclic fatty acids), amino acids, proteins (e.g., oligopeptide, polypeptide), low-molecular-weight compounds, compound libraries, random peptide libraries, natural ingredients (e.g., ingredients derived from microbes, animals and plants, marine organisms, and others), foods, and the like.

The inventive screening method can be of any type as long as it enables evaluation of whether a test substance suppresses the expression and/or activity of an ATF7IP-PDGFRB fusion polypeptide. Typically, the inventive screening method comprises the following steps:

(1) bringing a test substance into contact with a cell that expresses an ATF7IP-PDGFRB fusion polypeptide comprising the SETDB1-binding domain of ATF7IP and the transmembrane region and kinase domain of PDGFRB, and having kinase activity;
(2) determining whether the expression and/or activity of the fusion polypeptide is suppressed or not; and
(3) selecting the substance determined to suppress the expression and/or activity of the fusion polypeptide, as a cancer therapeutic agent.

At step (1), the cell expressing an ATF7IP-PDGFRB fusion polypeptide is brought into contact with the test substance. A test substance-free solvent (e.g., DMSO) can be used as a control. The contact can be effected in a medium. The medium is selected as appropriate depending on various factors including the type of the cell to be used, and examples include a minimum essential medium (MEM) supplemented with about 5-20% fetal bovine serum, a Dulbecco's modified eagle's medium (DMEM), RPMI1640 medium, and 199 medium. The culture conditions are also selected as appropriate depending on various factors including the type of the cell to be used, and for example, the pH of the medium is in the range of approximately from 6 to 8, the culture temperature is in the range of approximately from 30 to 40° C., and the culture time is in the range of approximately from 12 to 72 hours.

Examples of the cell expressing an ATF7IP-PDGFRB fusion polypeptide include, but are not limited to, cancer tissue-derived cells intrinsically expressing said fusion polypeptide, cell lines induced from said cells, and cell lines made by genetic engineering. Whether a cell expresses an ATF7IP-PDGFRB fusion polypeptide can also be confirmed using the inventive detection method described above. The cell is generally a mammalian cell, preferably a human cell.

At step (2), it is determined whether the test substance suppresses the expression and/or activity of said fusion polypeptide or not. The expression of a fusion polypeptide can be measured by determining the mRNA or protein level in a cell using a known analysis technique such as Northern blotting, quantitative PCR, immunoblotting, or ELISA. Also, the activity of a fusion polypeptide can be measured by a known analysis technique (e.g., kinase activity assay). The resulting measured value is compared with the value measured in a control cell not contacted with the test substance. The comparison between the measured values is made preferably based on the presence or absence of a significant difference. If the value measured in the cell contacted with the test substance is significantly lower than that measured in the control cell, it can be determined that the test substance suppresses the expression and/or activity of said fusion polypeptide.

Alternatively, since the cell expressing such a fusion polypeptide shows enhanced growth, the growth of said cell can be used as an indicator for the determination at this step. In this case, the growth of the cell contacted with the test substance is measured as a first step. The cell growth measurement can be made by a per se known technique such as cell count, ³H-thymidine incorporation, or BRDU. Next, the growth of the cell contacted with the test substance is compared with that of a control cell not contacted with the test substance. The growth level comparison is made preferably based on the presence or absence of a significant difference. The value for the growth of the control cell not contacted with the test substance can be a value measured prior to, or at the same time as, the measurement of the growth of the cell contacted with the test substance, and the value measured at the same time is preferred from the viewpoints of the accuracy and reproducibility of the test. If the results of the comparison show that the growth of the cell contacted with the test substance is suppressed, it can be determined that the test substance suppresses the expression and/or activity of said fusion polypeptide.

At step (3), the test substance determined to suppress the expression and/or activity of said fusion polypeptide at step (2) is selected as a cancer therapeutic agent.

Thus, the inventive screening method makes it possible to obtain a cancer therapeutic agent applicable to the treatment of patients having a responsible mutation for cancer which has been conventionally unknown.

<Isolated Fusion Polypeptide or Fragment Thereof, and Polynucleotide Encoding the Same>

The present invention provides an isolated ATF7IP-PDGFRB fusion polypeptide (hereinafter also referred to as the “inventive fusion polypeptide”) or a fragment thereof, which comprises the SETDB1-binding domain of ATF7IP and the transmembrane region and kinase domain of PDGFRB, and has kinase activity.

For the purpose of the present specification, the “isolated” substance refers to a substance substantially separated or purified from other substances (preferably, biological factors) found in an environment in which the substance naturally occurs (e.g., in a cell of an organism) (for example, if the substance of interest is a nucleic acid, the “other substances” corresponds to other factors than nucleic acids as well as nucleic acids containing other nucleic acid sequences than that of the nucleic acid of interest; and if the substance of interest is a protein, the “other substances” corresponds to other factors than proteins as well as proteins containing other amino acid sequences than that of the protein of interest). For the purpose of the specification, the term “isolated” means that a substance has a purity of preferably at least 75% by weight, more preferably at least 85% by weight, still more preferably at least 95% by weight, and most preferably at least 96% by weight, at least 97% by weight, at least 98% by weight, at least 99% by weight, or 100%. Examples of the “isolated” polynucleotide and polypeptide include not only polynucleotides and polypeptides purified by standard purification techniques but also chemically synthesized polynucleotides and polypeptides.

The “fragment” refers to a fragment of the inventive fusion polypeptide, which consists of a consecutive partial sequence comprising sequences upstream and downstream from the point of fusion. The sequence upstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) from the point of fusion to the N-terminus of the inventive fusion polypeptide. The sequence downstream from the point of fusion, as contained in said partial sequence, can comprise at least one amino acid residue (for example, at least 2, 3, 4, 5, 6, 7, 8, 9, 10, 20, 30, 40, 50 or 100 amino acid residues) from the point of fusion to the C-terminus of the inventive fusion polypeptide. The length of the fragment is not particularly limited, and is generally at least 8 amino acid residues (for example, at least 9, 10, 11, 12, 13, 14, 15, 20, 25, 50 or 100 amino acid residues).

Also, the inventive fusion polypeptide can be, for example, any of the isolated fusion polypeptides mentioned below:

(i) a polypeptide consisting of the amino acid sequence of SEQ ID NO: 2 or 4,
(ii) a polypeptide consisting of an amino acid sequence derived from the amino acid sequence of SEQ ID NO: 2 or 4 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2 or 4, and the polypeptide having kinase activity.

The meanings of the terms used in relation to the inventive fusion polypeptide are as defined above in <Specific responsible mutation for cancer>.

Further, the present invention provides an isolated polynucleotide encoding the inventive fusion polypeptide or the fragment thereof as described above (hereinafter also referred to as the “inventive polynucleotide”). The inventive polynucleotide can be in the form of any of mRNA, cDNA and genomic DNA. Also, the polynucleotides may be double- or single-stranded.

A typical example of the cDNA encoding the ATF7IP-PDGFRB fusion polypeptide is a polynucleotide consisting of the nucleotide sequence of SEQ ID NO: 1 or 3.

The inventive polynucleotide can be made by a per se known technique. For example, the inventive polynucleotide can be extracted using a known hybridization technique from a cDNA library or genomic library prepared from cancer tissues or the like harboring an ATF7IP-PDGFRB fusion polynucleotide. The inventive polynucleotides can also be prepared by amplification utilizing a known gene amplification technique (PCR), with mRNA, cDNA or genomic DNA prepared from the cancer tissues or the like being used as a template. Alternatively, the polynucleotides can be prepared utilizing a known gene amplification or genetic recombination technique such as PCR, restriction enzyme treatment, or site-directed mutagenesis (Kramer, W. & Fritz, H. J., Methods Enzymol., 1987, 154, 350), using, as starting materials, the cDNAs of wild-type genes from which to derive those segments of the ATF7IP-PDGFRB fusion polynucleotide which extend toward the 5′- or 3′-end.

The inventive fusion polypeptide or the fragment thereof can also be made by a per se known technique. For example, after such a polynucleotide prepared as mentioned above is inserted into an appropriate expression vector, the vector is introduced into a cell-free protein synthesis system (e.g., reticulocyte extract, wheat germ extract) and the system is incubated, or alternatively the vector is introduced into appropriate cells (e.g., E. coli, yeast, insect cells, animal cells) and the resulting transformant is cultured; in either of those ways, the inventive fusion polypeptide can be prepared.

The inventive fusion polypeptide or the fragment thereof can be used as a marker in the inventive detection method or the like, or can be used in other applications including preparation of an antibody against the inventive fusion polypeptide.

### EXAMPLES

Hereunder, the present invention will be more specifically described by way of working examples, but this invention is not limited to the examples given below.

### Example 1

### Samples

Total RNAs were extracted using the miRNeasy mini kit (Qiagen) from leukemic cells obtained from 300 children affected by pediatric leukemia (acute lymphoblastic leukemia). The present study was conducted with the approval by the institutional review boards of the institutions involved in the study.

<RNA Sequencing>

cDNA libraries for RNA sequencing were prepared using the TruSeq RNA sample preparation kit v2 Set B (Illumina) according to the manufacturer's standard protocol. The sequences of the total RNAs were analyzed by paired-end sequencing using a next-generation sequencer (HiSeq1000 Illumina).

<Detection of Fusion Transcripts>

Detection of fusion transcripts was performed using the DeFuse program (http://sourceforge.net/apps/mediawiki/defuse/index.php?title=DeFuse), a dedicated algorithm for fusion gene detection in cancer cells.

<RT-PCR and Sanger Sequencing>

cDNAs were synthesized using the ReverTra Ace® qPCR RT Master Mix (Toyobo) from the total RNAs extracted from leukemic cells from the subjects, and the resulting cDNAs were subjected to PCR amplification with rTaq DNA Polymerase (Toyobo). Agarose gel electrophoresis was performed to verify the amplification of gene fragments of desired size. The amplified gene fragments were TA-cloned into the pGEM-T Easy vector (Promega). Nucleotide sequencing was done using the BigDye® Terminator v3.1 Cycle Sequencing Kit (Applied Biosystems) and the ABI 3130x1 Genetic Analyzer (Applied Biosystems), with the T7 and SP6 sequences located respectively toward the 5′- and 3′-ends of the multicloning site of this vector being used as primers.

This example describes the identification of a novel fusion transcript in leukemic cells.

In order to identify a novel fusion transcript potentially serving as a target for treatment, transcriptome analysis was carried out on the leukemic cells obtained from 300 children with pediatric leukemia (acute lymphoblastic leukemia).

As a result of the analysis of the paired-end reads obtained by the RNA sequencing, the novel fusion gene ATF7IP-PDGFRB conceivably produced by a chromosomal translocation (t(5;12)), as shown in FIG. 1, was identified in one subject.

Further, RT-PCR was performed using the following primers on the sample with the detected ATF7IP-PDGFRB gene, and the obtained PCR product was subjected to sequence analysis to verify the expression of this novel fusion gene (FIGS. 2 and 3).

Forward primer:
(SEQ ID NO: 11)
CAAGTGGACCATCTCAGACCAC
Reverse primer:
(SEQ ID NO: 12)
ATTTAAGCATCTTGACGGCCAC

The ATF7IP-PDGFRB gene is a fusion gene between the ATF7IP gene located at chromosome 12p13.1 and the PDGFRB gene located at chromosome 5q33.1.

These results demonstrated that the gene fusion discovered in Example 1 can be detected by verifying the presence or absence of the amplification of a specific band by RT-PCR and determining the nucleotide sequence of a PCR product.

Additionally, the gene expression pattern displayed in the ATF7IP-PDGFRB gene-positive subject was a Ph-like phenotype, i.e., a pattern in which genes are expressed in a similar profile to BCR-ABL fusion gene-positive leukemia (Ph1-ALL), a poor-prognosis subgroup of pediatric leukemia but no BCR-ABL fusion gene is detected; thus, it was considered that the ATF7IP-PDGFRB gene-positive subject may belong to a peculiar poor-prognosis subgroup of acute lymphoblastic leukemia. Therefore, it was suggested that detection of the ATF7IP-PDGFRB gene may make it possible to identify a previously unknown poor-prognosis subgroup of ALL.

### Example 2

This example describes a monotherapy with the tyrosine kinase inhibitor (TKI) dasatinib in a patient with recurrent Ph-like acute lymphoblastic leukemia bearing ATF7IP/PDGFRB translocation.

Case Report

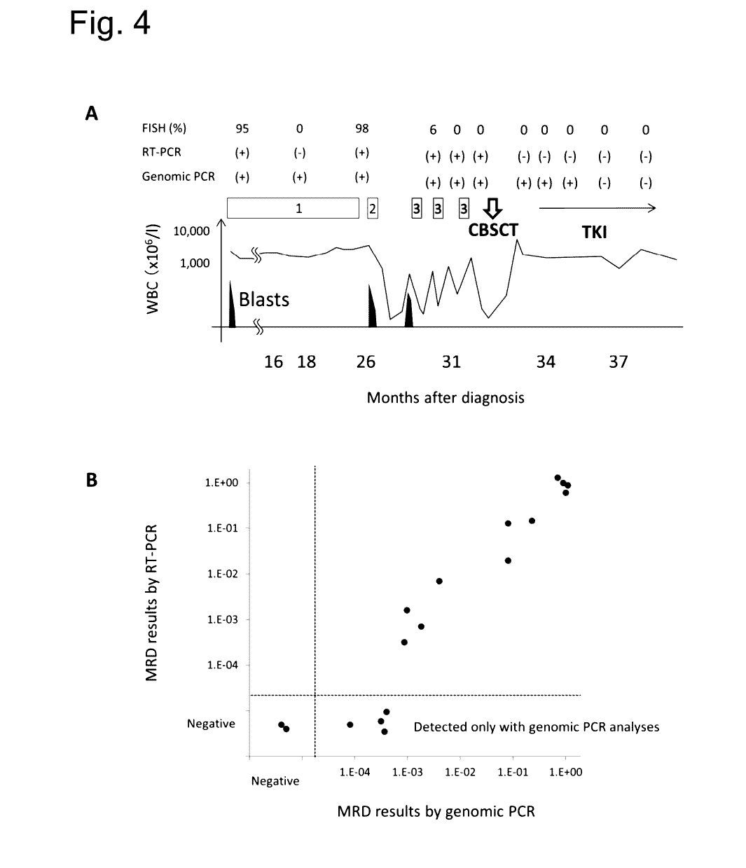

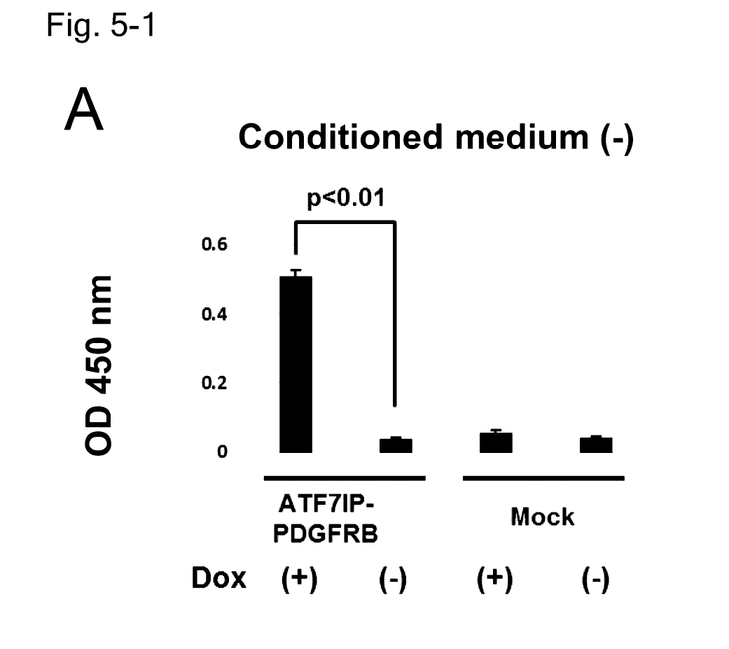

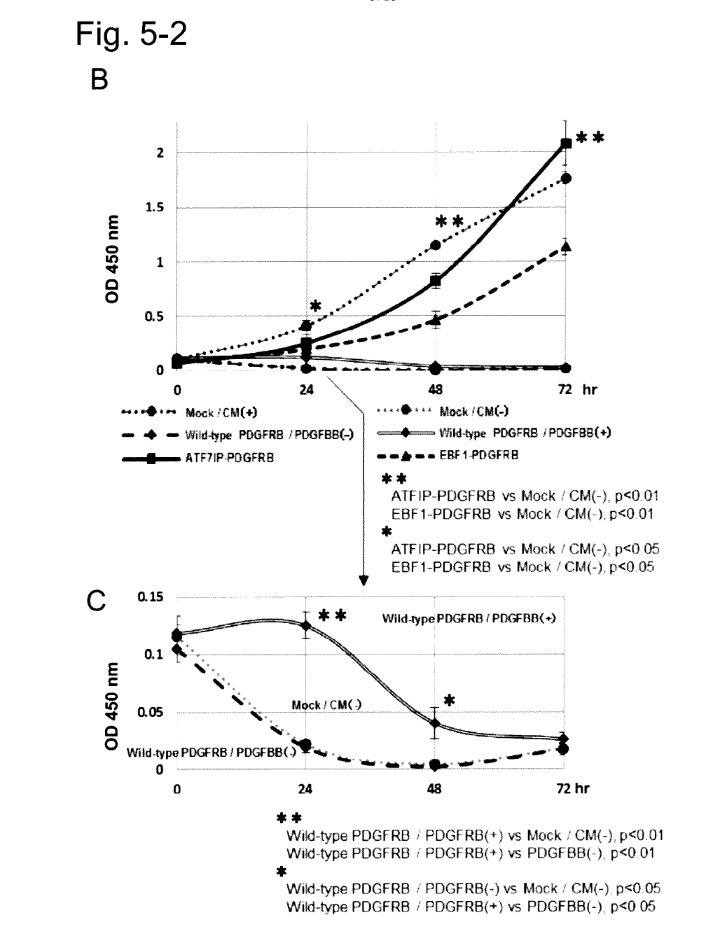

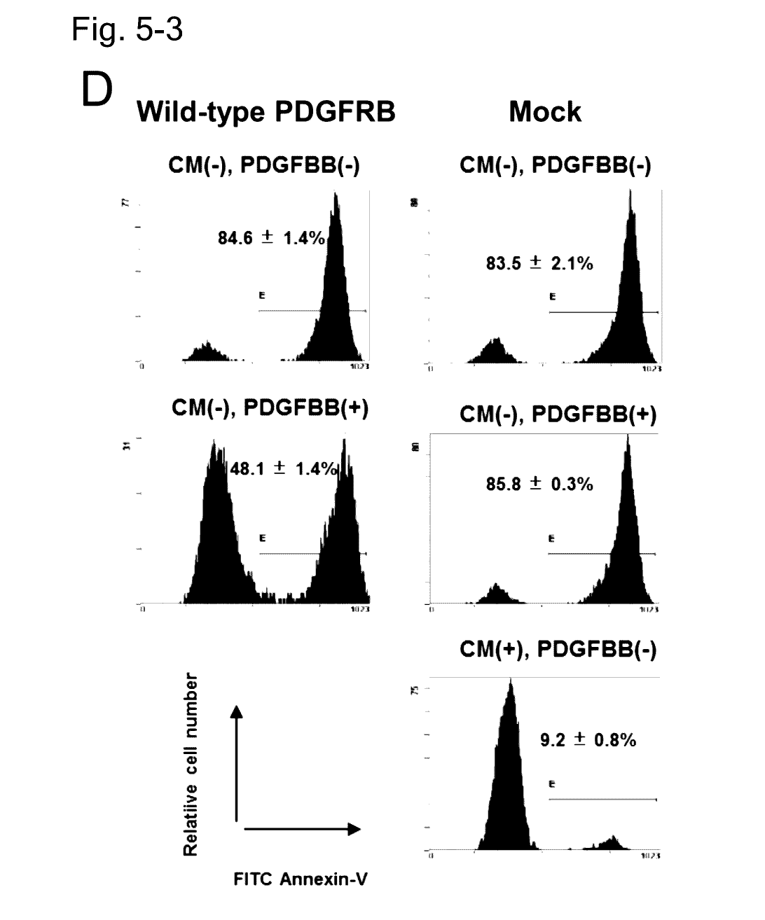

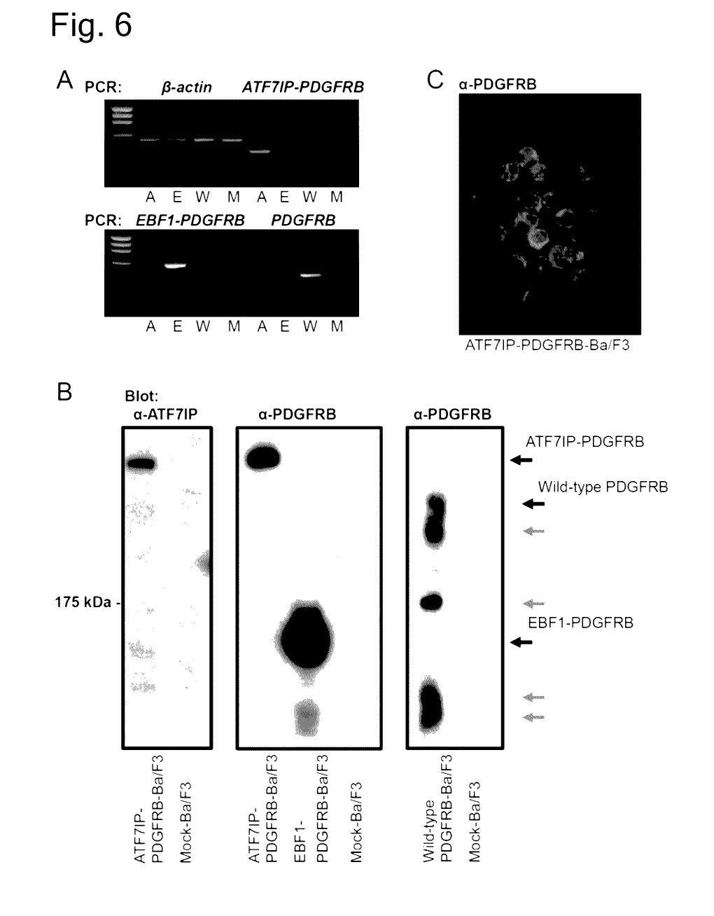

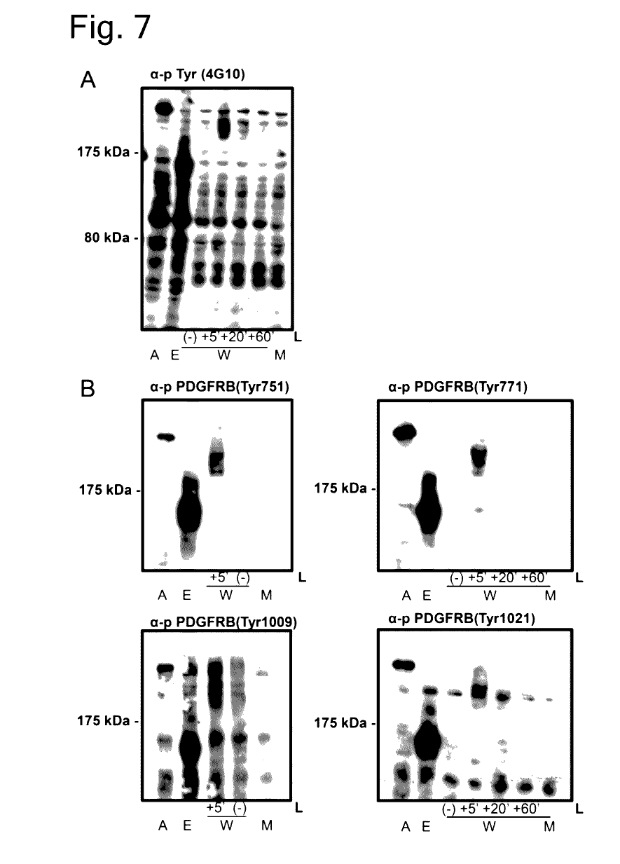

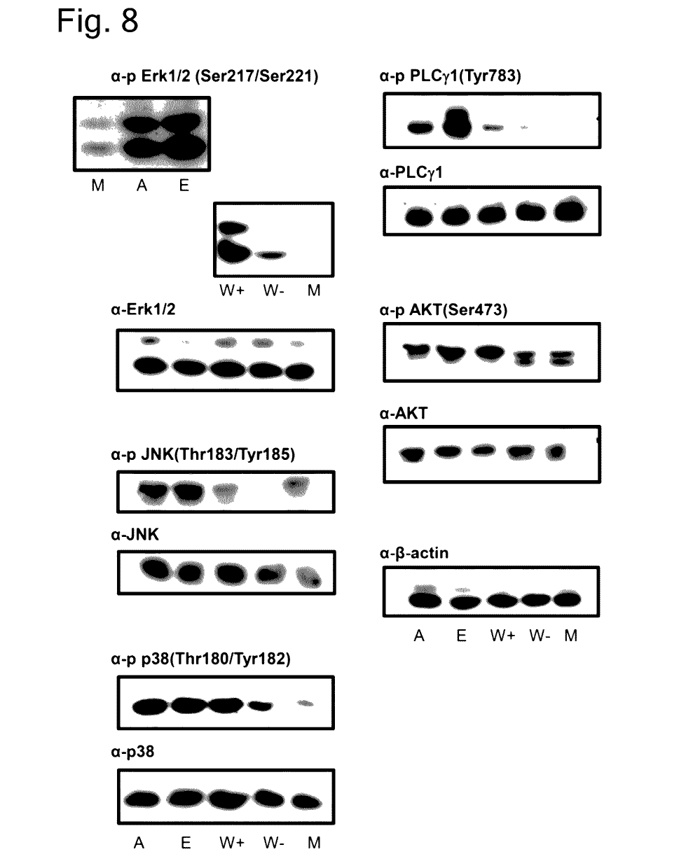

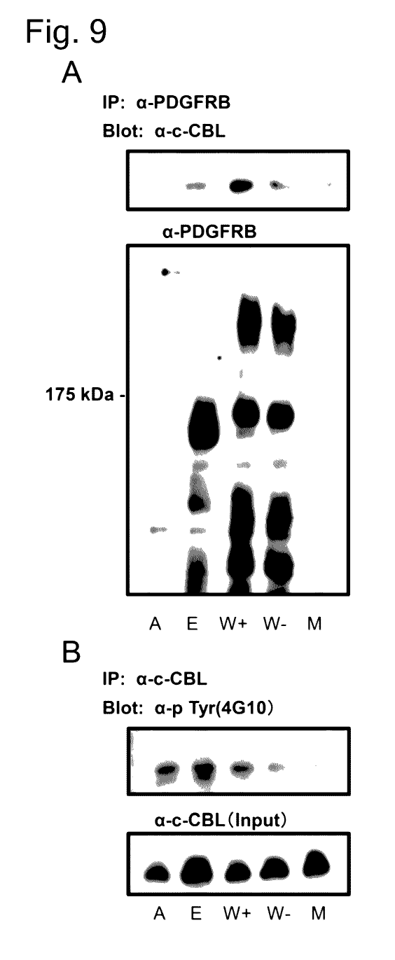

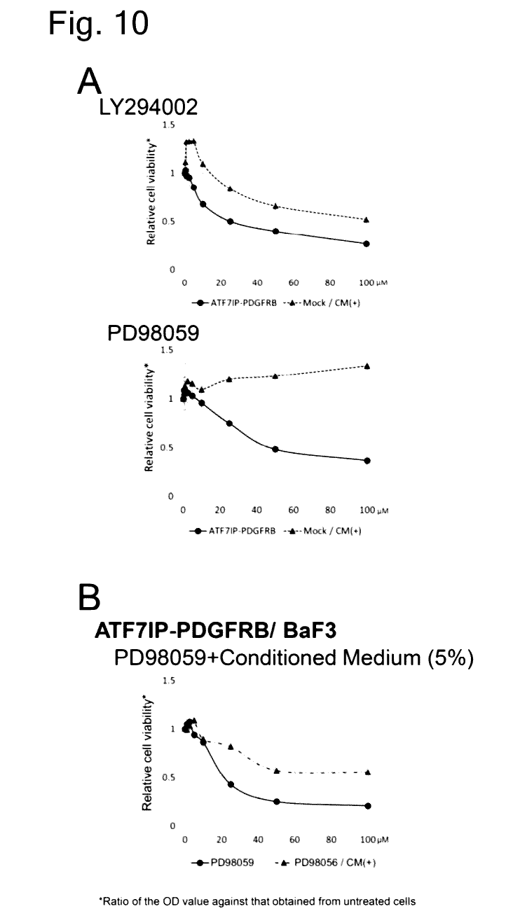

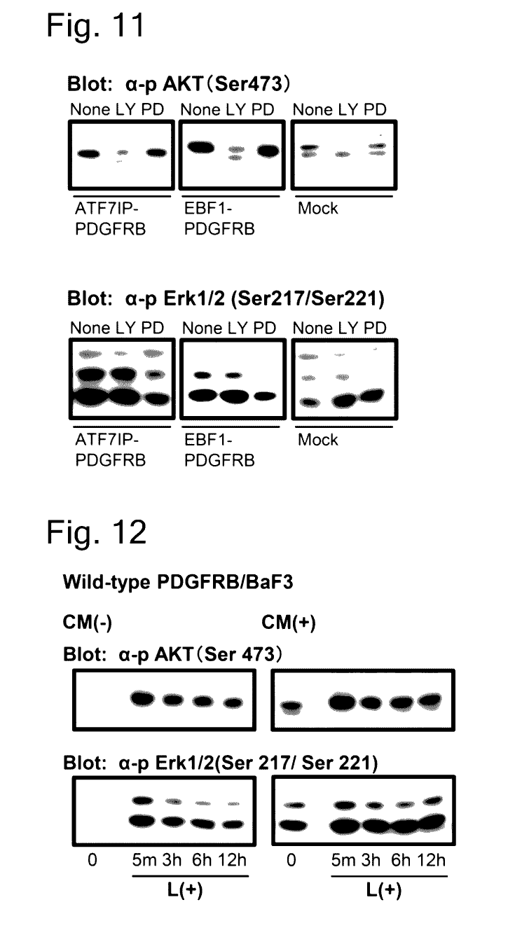

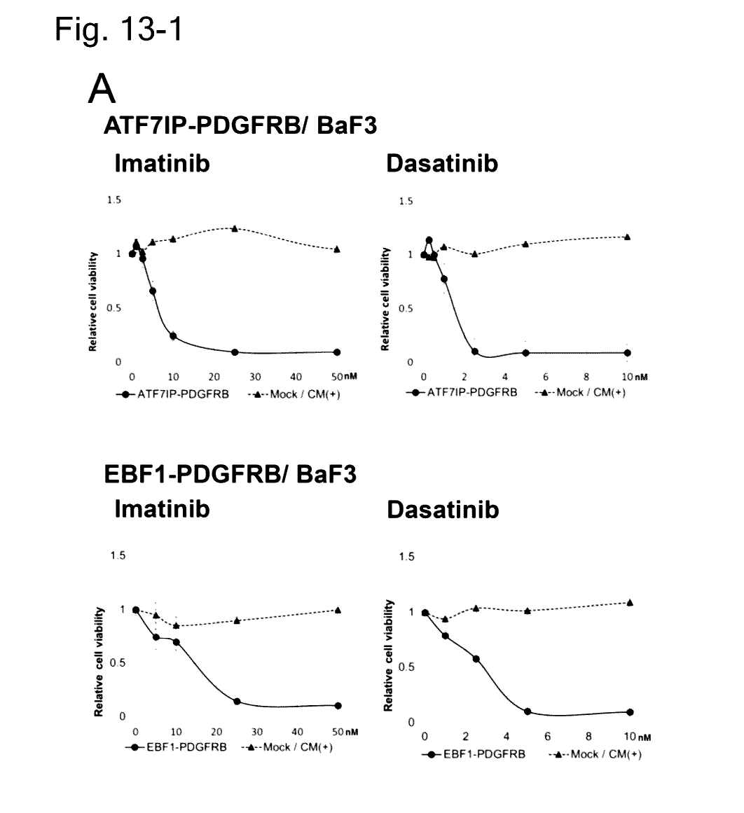

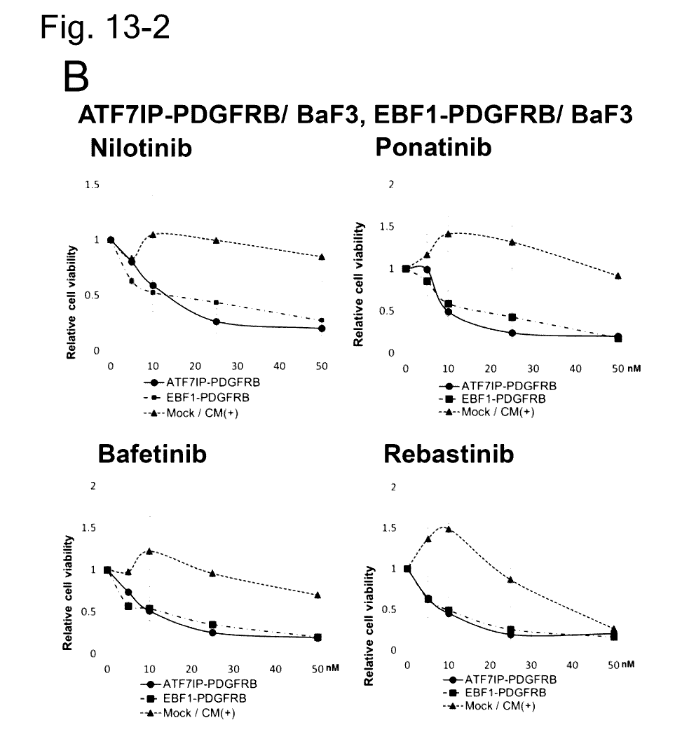

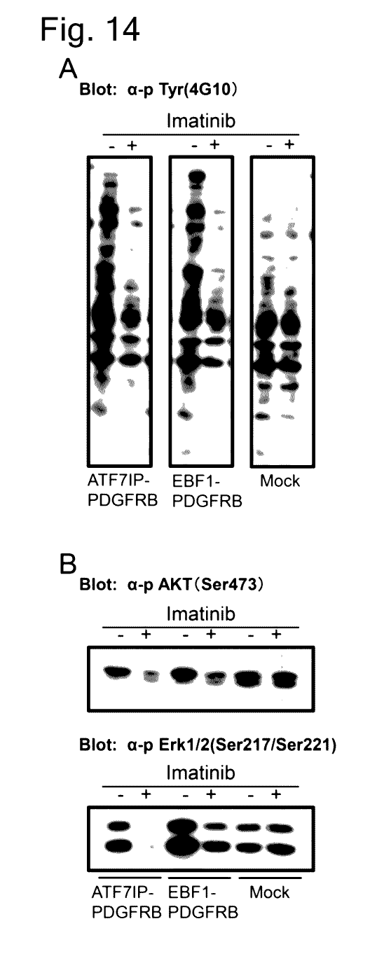

On February 2011, an 8-year-old male child developed standard-risk ALL. The child successfully completed induction and consolidation therapies, but experienced a relapse at 26 months after the diagnosis, even while receiving maintenance chemotherapy. The observation of ATF7IP/PDGFRB by both fluorescence in situ hybridization (FISH) and RT-PCR analyses provided an evidence to confirm the recurrence of the original clone (FIG. 4A). The RT-PCR performed 8 months before the relapse showed a negative result for minimum residual disease (MRD) (Kobayashi K., et al., Br J Haematol, 2014, 165, 836-841); thus, it was recognized that molecular monitoring with RT-PCR is not sensitive enough to predict an early relapse. Therefore, it was decided to establish an alternative MRD monitoring approach, and to perform genome mapping to search for a fusion sequence in the ATF7IP/PDGFRB translocation. A genomic breakpoint was identified between ATF7IP intron 13 and PDGFRB exon 10, and a primer pair targeting a leukemia-specific DNA fingerprint was established. It is of particular note that the leukemia-specific DNA sequence was readily detected even from the remission sample obtained at 18 months after the diagnosis (FIG. 4A). This suggests that genomic PCR-based monitoring of MRD, even when it is detected only at a very low level, is a powerful predictor of early relapse.

The relapsing blast cells showed complete refractoriness to the ALL-type protocol with dexamethasone, vincristine, cytarabine, methotrexate, and L-asparaginase. Thus, the therapy was switched to a reinduction AML-type regimen with etoposide, cytarabine, and mitoxantrone (Tsukimoto I., et al., J Clin Oncol, 2009, 27, 4007-4013). Following 3 courses of the therapy, cytogenic remission was achieved, but MRD was still detected within the quantitative range. The child consecutively received cord blood stem cell transplantation (CBSCT) followed by a myeloablative conditioning regimen with etoposide (50 mg/kg/day on day −4) and melphalan (70 mg/m²/day on days −3 and −2), along with total body irradiation (12 Gy). The subsequent clinical course was uneventful with a major molecular response based on RT-PCR analysis, but the fusion-specific leukemic DNA sequence was persistently detected with genomic PCR even at 34 months after the initial diagnosis. The cut-off value of MRD for predicting relapse was largely unknown; however, as was evidenced in the former clinical analyses, the presence of leukemic DNA even in the molecular remission sample suggested that the child was at high risk of impending relapse. Thus, it was decided to initiate a therapy with the second-generation TKI dasatinib (66 mg/m²/day) for the purpose of eradication of the MRD. The therapeutic response was prompt, with the MRD disappearing within 3 months according to the leukemia-specific genomic PCR analysis (FIG. 4A). The child had remained in complete molecular remission at least for 12 months.

In order to confirm the clinical outcome of TKI monotherapy for Ph-like ALL, it is necessary to perform longitudinal disease monitoring with durable sensitivity and specificity. In fact, RT-PCR analysis of the pathognomic fusion transcript, i.e., BCR/ABL, is commonly used for the therapeutic monitoring of TKI (Hughes T., et al., Blood, 2006, 108, 28-37), but it has also been shown that RT-PCR negativity does not directly correlate with the clinical outcome in CML (Chu S., et al., Blood, 2011, 118, 5565-5572). For the development of personalized therapy on hematological malignancy, a detailed analysis of MRD is not only important for the diagnosis but also for the justification of TKI therapy. In this regard, it is particularly important to note that recent increasing evidences suggest that genomic PCR targeting a junctional DNA sequence is more sensitive than conventional RT-PCR in adult CML patients (Mattarucchi E., et al., J Mol Diagn, 2009, 11, 482-487; Bartley P. A., et al., Int J Lab Hematol, 2010, 32, e222-228), but its clinical significance in BCP-ALL is largely unknown. For that reason, a paired comparison of MRD detection sensitivity between RT-PCR and genomic PCR was performed using 17 samples obtained from the patient at different points of time (FIG. 4B). In the 11 samples, in which MRD levels were detectable within the quantitative range of the PCR assay, there was observed a clear correlation in results between the two methods. Conversely, discordant results were found in 4 samples; namely, leukemic DNA was detected even from the molecular-remission samples with negative RT-PCR results. In contrast, no sample was found in which MRD was positive by RT-PCR but negative by genomic PCR. Thus, it is considered that the discordance in results between RT-PCR and genomic PCR analyses is likely to be mostly due to a difference in detection sensitivity rather than sample variations. Furthermore, it is possible to speculate that the fusion transcript level examined with RT-PCR is less closely related to the number of residual leukemic cells due to various reasons such as transcriptional silencing, especially with a quiescent cellular status, and possible involvement of mRNA degradation (Shibata Y., et al., Genome Medicine, 2010, 2, 70). In contrast, genomic PCR is directly related to the measurement of the number of leukemic cells, as each leukemic cell contains one copy of the fusion DNA sequence. Since DNA is more stable than RNA and does not require a reverse transcription step, a DNA-based MRD analysis may also facilitate a retrospective research, as was shown in the present case. Furthermore, Pagani, et al. reported that fusion-specific DNA can be detected in 30% or more of RT-PCR-negative CML patients on long-term TKI treatment (Pagani I. S., et al., Oncoscience, 2014, 1, 510-521). The report stressed that the genomic PCR targeting the BCR/ABL genomic breakpoint could be utilized in combination with conventional techniques for the long-term monitoring of CML patients with TKI therapy. Thus, the above-mentioned observations including those obtained in this example collectively suggest that genomic PCR targeting a fusion sequence can also be utilized as one of the reliable supplementary techniques for MRD monitoring in Ph-like ALL patients receiving TKI therapy.

Discussion

The advent of TKI has been of marked therapeutic benefit, not only for CML patients, but also for patients with other rare types of pediatric hematological malignancy, such as Ph-like ALL (Roberts K. G., et al., N Engl J Med, 2014, 371, 1005-1015; Weston B. W., et al., J Clin Oncol, 2013, 31, e413-416; Lengline E., et al., Haematologica, 2013, 98, e146-148). Furthermore, it is of particular note that patients with myeloid malignancies bearing PDGFRB fusion genes show excellent therapeutic outcomes in response to the TKI imatinib mesylate, with a 10-year overall survival rate of 90% without secondary resistance (Cheah C. Y., et al., Blood, 2014, 123, 3574-3577). Thus, it is believed that companion diagnostics focusing on molecular targets, such as the PDGFRB fusion gene, may facilitate future personalized cancer therapy for children.

### Example 3

This example describes that the ATF7IP-PDGFRB fusion kinase induces cell transformation.

Materials and Methods

Reagents

The TKIs used were imatinib, dasatinib, nilotinib (LC Laboratories, Woburn, Mass., USA), ponatinib, rebastinib, and bafetinib (Selleck, Houston, Tex., USA). The mitogen-activated protein kinase kinase (MEK) inhibitor, PD98059, and the phosphatidylinositol 3-kinase (PI3 kinase) inhibitor, LY294002, were purchased from Merck Millipore (Darmstadt, Germany).

Plasmid Construction

The cDNA of wild-type (WT-) PDGFRB was purchased from Promega Corporation (Madison, Wis., USA) as Flexi® ORF clone pF1KB7023 (kazusa clone cp01083). The coding region was isolated by digestion with the Sfg I and Pme I enzymes followed by blunt ending with a T4 DNA polymerase. After the attachment of protruding dATP to the 3′-end by an rTaq polymerase (Toyobo Co., Ltd., Osaka, Japan), WT-PDGFRB cDNA was subcloned into the pGEM-T Easy or pGEM-T vector (Promega) in the reverse direction (from SP6 to T7 site).

The use of the clinical materials for the research was approved by the institutional review boards, and informed consent was obtained from the parents or guardians. A 5′ portion of ATF7IP cDNA contained in the ATF7IP-PDGFRB chimeric gene was amplified from a clinical specimen of the patient carrying the fusion gene, by PCR with KOD plus ver. 2 (Toyobo) using the following primers, with said portion being divided into three parts, ATF7IP-1, -2, and -3:

ATF7IP-1 forward (Fwd):
(SEQ ID NO: 13)
5′-TTCAGAATGGACAGTTTAGAAGAACCTCAG-3′,
ATF7IP-1 reverse (Rev):
(SEQ ID NO: 14)
5′-TCTGCTGGAGAGCACGTTTC-3′,
ATF7IP-2 Fwd:
(SEQ ID NO: 15)
5′-AAGCTTGCACCTTCTGAGGATGA-3′,
ATF7IP-2 Rev:
(SEQ ID NO: 16)
5′-ATCGGATCTCGTAACGTGGC-3′,
ATF7IP-PDGFRB-3 Fwd:
(SEQ ID NO: 17)
5′-TGGTCCTCTCTGATGAAGAGGATATTTC-3′,
and
ATF7IP-PDGFRB-3 Rev:
(SEQ ID NO: 18)
5′-TCATCGTGGCCTGAGAATGG-3′.

The PCR products having protruding dATP attached to the 3′-end as described above were each subcloned into the pGEM-T Easy vector (Promega) in the forward direction (from T7 to SP6 site), as described previously (Tomita, et al., Leukemia Research, 2014, 38, 361-370). To obtain the full-length ORF of ATF7IP-PDGFRB cDNA, the cDNA fragments ATF7IP-3, -2 and -1 digested respectively with the following combinations, Not I/Pmi I, Not I/Afi I, and Not I/Cla I, were subcloned into pGEM-T-WT-PDGFRB by sequential ligation.

Likewise, a 5′ portion of EBF1 cDNA was amplified using the following primers:

EBF1-PDGFRB Fwd:
(SEQ ID NO: 19)
5′-ATGTTTGGGATTCAGGAAAGCATCC-3′,
and
EBF1-PDGFRB Rev:
(SEQ ID NO: 20)
5′-ATGCGGTAACCCCGTTTGAT-3′.

The PCR products were subcloned into the pGEM-T Easy vector. The full length ORF of EBF1-PDGFRB cDNA was obtained by ligation of the Sph I (blunted) and Bam HI fragments of EBF1 cDNA with the NdeI (blunted) and Bam HI fragments of pGEM-T-WT-PDGFRB.

To obtain a retroviral expression vector, the full-length inserts of the cDNAs of the chimeric gene were digested with the Sph I (blunted) and Not I enzymes and subcloned into the Nru I and Not I sites of the pRetroX Tight vector (Clontech Laboratories, Inc., Madison, Wis., USA). In the case of WT-PDGFRB, the full-length insert of the cDNA was isolated by Eco RI digestion and subcloned into the Eco RI site in the pRetroX Tight vector.

The expressions of the ATF7IP-PDGFRB, EBF1-PDGFRB, and WT-PDGFRB genes were amplified from cDNAs prepared from Ba/F3 cells transfected with said genes, by means of PCR using an rTaq polymerase (Toyobo), as described previously (Iijima, et al., Eur J Immunol, 2012, 42, 3405-3415). The amplification was performed using the following primers:

ATF7IP-PDGFRB detection Fwd-1:
(SEQ ID NO: 21)
5′-CGTGAATGTAACACATCGTCCA-3′,
ATF7IP-PDGFRB detection Rev-1:
(SEQ ID NO: 22)
5′-AGCTCCCACGTGGAGTCATAG-3′,
EBF1-PDGFRB detection Fwd-1:
(SEQ ID NO: 23)
5′-GGGCGTGAATTCGTTCAGTG-3′,
EBF1-PDGFRB detection Rev-1:
(SEQ ID NO: 24)
5′-ATCGGATCTCGTAACGTGGC-3′,
WT-PDGFRB detection Fwd-1:
(SEQ ID NO: 25)
5′-TCCAGCACCTTCGTTCTGAC-3′,
and
WT-PDGFRB detection Rev-1:
(SEQ ID NO: 26)
5′-CCAGAAAAGCCACGTTGGTG-3′.

As an internal control, the primer set for the detection of mouse β-actin (Agilent Technologies, Santa Clara, Calif., USA) was also used.

Cells, Cell Culture, and Transfection

Ba/F3 cells and WEHI-3 cells (RIKEN Bioresource Center, Tsukuba, Ibaraki, Japan) were maintained, as described previously (Tomita, et al., Leukemia Research, 2014, 38, 361-370). In the case of Ba/F3 cells, 10% (vol/vol) conditioned medium prepared from the WEHI-3 cell line containing IL-3 was supplemented for maintenance, as described previously (Tomita, et al., Leukemia Research, 2014, 38, 361-370).

Chimera gene-inducible cell lines were generated by employing retroviral transfection using the Retro-X™ Tet-On® Advanced Inducible Expression System and Retro-X™ Universal Packaging System (Clontech), basically following a previously described procedure (Iijima, et al., Eur J Immunol, 2012, 42, 3405-3415). In these systems, the Tet-on Advanced vector carrying the neomycin-resistant gene and the pRetroX Tight expression vector carrying the puromycin-resistant gene were used. In a total of 3 mL of the medium, 6×10⁵ Ba/F3 cells transduced with the Tet-on Advanced vector were further infected with the pRetroX Tight retrovirus containing either ATF7IP-PDGFRB or EBF1-PDGFRB, and exposed to this retrovirus for 10 hours. The cells were then washed and divided into two aliquots, which were treated with neomycin and puromycin for drug selection in the presence or absence of 1 μg/mL of doxycycline (DOX), whereby the expression of the intended proteins was induced. After 24-hour incubation, the cells were washed again to withdraw the WEHI-3-conditioned medium. Half of each aliquot of treated cells was plated for cell proliferation assay in triplicate, and the remaining half was subjected to sequential drug selection. The stable drug-resistant cells were cloned by repeating limiting dilution 3 times and used in the following experiments. A WT-PDGFRB or Mock transfectant bearing an empty vector was established in the same way, except that WEHI-3 conditioned medium was left present consistently as the transfectant cannot proliferate in an IL-3-independent manner.

Cell Proliferation Assay

The chimeric gene transfectants and Mock Ba/F3 cells were each washed twice with RPMI-1640 medium, dispensed in triplicate into 96-well plates (2.5×10⁴ cells/0.1 mL per well) in the presence or absence of the reagents, and cultured at 37° C. for the periods indicated in the figures. Cell proliferation was analyzed using water-soluble tetrazolium salt (WST) assay (Cell Counting Kit-8, Dojindo, Kumamoto, Japan), as described previously (Yamada, et al., Int J Hematol, 2013, 97, 73-82). The data were analyzed by F-test. P values less than 0.05 were considered significant. All experiments were performed at least three times to calculate means and SEM values.

Flow Cytometric Analysis and Immunocytochemistry

The frequency of apoptotic cells was quantified using the MEBCYTO® Apoptosis Kit (Medical and Biological Laboratories, Nagoya, Japan), and then analyzed according to the manufacturer's protocol, as described previously (Tomita, et al., Leukemia Research, 2014, 38, 361-370).

For immunocytochemistry, the cells were treated with the PerFix-nc Kit (Beckman Coulter, Inc., Indianapolis Ind., USA) according to the manufacturer's protocol and stained with a combination of rabbit monoclonal anti-PDGFRB antibody (Ab) (Cell Signaling Technology, Inc., Danvers, Mass., USA) and Alexa Fluor® 546 goat anti-rabbit IgG (Life Technologies, Waltham, Mass., USA). The cells were mounted on a slide glass, counterstained with 4′,6-diamidino-2-phenylindole (DAPI) using Fluoroshield Mounting Medium with DAPI (Immunobioscience Corp., Mukilteo, Wash., USA), and visualized by DeltaVision Elite (GE Healthcare Life Sciences, Buckinghamshire, UK).

Immunoblotting and Immunoprecipitation

Immunoblot analysis was performed as described previously (Kiyokawa, et al., J Biol Chem, 1997, 272, 18656-18665; Tomita, et al., Leukemia Research, 2014, 38, 361-370) using the following antibodies: anti-phosphotyrosine (4G10; Merck Millipore), anti-PDGF receptor β (28E1), anti-phospho-PDGF receptor β Tyr751 (C63G6), anti-phospho-PDGF receptor β Tyr 771 (76D6), anti-phospho-PDGF receptor β Tyr1009 (42F9), anti-phospho-PDGF receptor β Tyr1021 (6F10), anti-phospho-p44/42 MAPK (ERK1/2, Tyr202/Tyr204), anti-phospho-p38 MAPK (Thr180/Tyr182), anti-phospho-JNK (Thr183/Tyr185, 81E11), anti-phospho-AKT (Ser473), anti-phospho-PLCγ1 (Tyr783), anti-Casitas B-lineage lymphoma binding (Cbl), anti-p38 MAPK antibody and anti-JNK antibody (Cell Signaling Technology); anti-ATF7IP (MCAF1) (Abcam, Cambridge, UK); anti-ERK, anti-PLCγ, (BD Biosciences, San Jose, Calif., USA); anti-β-actin (Sigma-Aldrich, St. Louis, Mo.); and horseradish peroxidase (HRP)-conjugated secondary antibody (Dako, Glostrup, Denmark). A 50-μg sample of each cell lysate was electrophoretically separated on an SDS-polyacrylamide gel and transferred to a nitrocellulose membrane (Amersham Hybond-ECL; GE Healthcare), as described previously (Kiyokawa, et al., J Biol Chem, 1997, 272, 18656-18665). The membranes were incubated with an appropriate combination of primary and secondary antibodies, washed, and detected with an enhanced chemiluminescent reagent system (ECL) and Western Blotting Detection Reagents (GE) (Kiyokawa, et al., J Biol Chem, 1997, 272, 18656-18665).

PDGFRB and chimeric proteins were immunoprecipitated from 1 mg of cell lysate prepared from each Ba/F3 transfectant, with the use of a combination of appropriate antibodies and protein G-agarose (F. Hoffmann-La Roche Ltd., Basel, Schweiz), as described previously (Kiyokawa, et al., J Biol Chem, 1997, 272, 18656-18665). Immunoprecipitates were separated on an SDS-polyacrylamide gel and then subjected to immunoblot analysis as described above using appropriate combinations indicated in the figures.

Results

The introduction of PDGFRB-related chimeric molecules induces IL-3-independent proliferation of Ba/F3 cells, but the introduction of WT-PDGFRB molecules does not induce such proliferation.

Firstly, a tetracycline-inducible gene expression system was used to examine whether the expression of chimeric molecules gives rise to IL-3-independent proliferation in Ba/F3 cells. After transfection with an ATF7IP-PDGFRB-expression vector, each aliquot of the Ba/F3 cells was treated in the presence or absence of DOX, and then WEHI-3-conditioned medium containing IL-3 was withdrawn. As shown in FIG. 5A, the one aliquot of Ba/F3 transfectants treated in the presence of DOX survived and grew in the absence of the conditioned medium while the other aliquot of Ba/F3 transfectants treated in the absence of DOX was unable to survive without the conditioned medium; consequently, selection of ATF7IP-PDGFRB transfectants was readily achieved. In contrast, the Mock-transfected Ba/F3 cells were unable to survive without the conditioned medium independently of DOX treatment. Similarly, the Ba/F3 cells transfected with EBF1-PDGFRB also exhibited IL-3-independent proliferation after DOX treatment, whereas the WT-PDGFRB transfectants did not (data not shown). After the cloning of the transfectants, it was further confirmed that the ATF7IP-PDGFRB and EBF1-PDGFRB transfectants showed cell proliferation even in the absence of the conditioned medium containing IL-3, whereas the Mock-transfected Ba/F3 cells were able to proliferate only in the presence of the conditioned medium (FIG. 5B). The growth rates of the PDGFRB-related chimera transfectants were equivalent to that of parental Ba/F3 cells growing in the presence of the conditioned medium (data not shown). The WT-PDGFRB transfectants were unable to survive without the conditioned medium even if the ligand PDGFBB was present (FIG. 5B). Interestingly, the addition of PDGFBB partially prolonged the survival of the WT-PDGFRB transfectants in the absence of the conditioned medium, as assessed by WST and Annexin V assays (FIGS. 5C & D).

Next, it was confirmed that the above-described effects were actually mediated by the introduced PDGFRB-related chimeric molecules. As shown in FIGS. 6A & B, appropriate mRNA and protein expressions in each of the transfectants were observed by RT-PCR and immunoblot analyses. As shown in FIG. 6C, immunocytochemistry revealed the cytoplasmic localization of ATF7IP-PDGFRB chimeric proteins.

Intracellular Cellular Signaling Mediated by PDGFRB-Related Chimeric Molecules in Ba/F3 Cells

Next assessed was the molecular basis of the IL-3-independent cell proliferation due to the expression of PDGFRB-related chimeric molecules. Since PDGFRB is a receptor tyrosine kinase, the state of tyrosine phosphorylation of cellular proteins and PDGFRB-related chimeric molecules themselves was examined. As shown in FIG. 7A, intracellular proteins were tyrosine-phosphorylated in the ATF7IP-PDGFRB- and EBF1-PDGFRB-expressing Ba/F3 cells, but not in the Mock cells. As for the case of WT-PDGFRB, stimulation with PDGFBB induces the tyrosine phosphorylation of WT-PDGFRB proteins. Also examined was the tyrosine phosphorylation of the particular residues located in PDGFRB. As shown in FIG. 7B, the Tyr751, Tyr771, Tyr1009, and Tyr1021 residues on both ATF7IP-PDGFRB and EBF1-PDGFRB were phosphorylated. In addition, the same tyrosine residues on WT-PDGFRB were phosphorylated after PDGFBB stimulation (FIG. 7B).

Next examined was the phosphorylation of molecules located downstream of PDGFRB. Upon phosphorylation, the Tyr751 residue on PDGFRB allowed the binding and activation of PI3 kinase and Nckβ (Kazlauskas, et al., Mol Cell Biol, 1992, 12, 2534-2544; Panayotou, et al., EMBO J, 1992, 11, 4261-4272; Nishimura, et al., Mol Cell Biol, 1993, 13, 6889-6896). Consistent with the phosphorylation of Tyr751 on PDGFRB, Ser473 on AKT, a downstream molecule of PI3 kinase, as well as Thr183/Tyr185 and Thr180/Tyr182 located respectively on c-Jun N-terminal kinase (JNK) and p38 MAPK, which are downstream molecules of Nckβ, were phosphorylated in the ATF7IP-PDGFRB- and EBF1-PDGFRB-transduced Ba/F3 cells unlike in the Mock cells, as assessed by immunoblot analysis (FIG. 8). In the WT-PDGFRB-transduced Ba/F3 cells, the same serine residue on AKT was also phosphorylated after stimulation with PDGFBB (FIG. 8). Similarly, the phosphorylation of the Tyr771 residue on PDGFRB allowed the binding and activation of RasGap (Kashishian, et al., EMBO J, 1992, 11, 1373-1382; Kazlauskas, et al., Mol Cell Biol, 1992, 12, 2534-2544). Consistent with the phosphorylation of the Tyr771 residue on PDGFRB, the phosphorylation levels of the Ser217/Ser221 residues on p44/42 MAP kinases (Erk1/2), downstream molecules of the Ras signaling pathway, revealed increased phosphorylation levels in both the two types of Ba/F3 cells containing PDGFRB-related chimeric kinases, like in WT-PDGFRB stimulated with PDGFBB (FIG. 8). Consistent with the phosphorylation of Tyr1009 and 1021 on PDGFRB, which allow the activation of PLC-γ (Reddi, et al., 2007), the Tyr783 residue on PLC-γ was phosphorylated in the Ba/F3 transfectants containing PDGFRB-related chimeras. Interestingly, the phosphorylation of the same residue on PLC-γ in the WT-PDGFRB-expressing cells was not significant even after stimulation with PDGFBB (FIG. 8).

Phosphorylation of c-Cbl and Binding of c-Cbl to PDGFRB in Ba/F3 Cells

As shown in FIG. 6B, immunoblot analysis of WT-PDGFRB revealed some unexpected bands of WT-PDGFRB protein with smaller molecular weights besides the predicted size, independently of PDGFBB stimulation, whereas the same test of ATF7IP- and EBF1-PDGFRB showed unique bands to the respective chimeric kinases. Since it has been documented that various growth factor receptors, including PDGFRB, undergo ligand-binding-mediated polyubiquitination and are thought to play a negative regulatory role in mitogenic signaling (Mori, et al., J Biol Chem, 1993, 268, 577-583; Waterman, et al., J Biol Chem, 1999, 274, 22151-22154), the above-described observation indicates the polyubiquitination of WT-PDGFRB in Ba/F3 cells. In addition, it has been reported that c-Cbl is responsible for the ubiquitination of PDGFRB (Miyake, et al., J Biol Chem, 1999, 274, 16619-16628), while ETV6-PDGFRB, a PDGFRB-related fusion protein detected in myeloproliferative neoplasms (MPNs), can escape from c-Cbl-induced ubiquitination and degradation (Toffalini F., et al., Haematologica, 2009, 94, 1085-1093). Therefore, the relationship between chimeric PDGFRB proteins and c-Cbl was examined. As shown in FIG. 9, immunoprecipitation and immunoblot analyses revealed that tyrosine residues on c-Cbl were phosphorylated in both ATF7IP- and EBF1-PDGFRBs as well as in PDGFBB-stimulated WT-PDGFRB. On the other hand, significant binding of c-Cbl protein to WT-PDGFRB after PDGFBB stimulation was observed, while ATF7IP- and EBF1-PDGFRB presented only limited binding of c-Cbl protein (FIG. 9). The data indicate that WT-PDGFRB activates c-Cbl and then is polyubiquitinated by it upon ligand stimulation, whereas ATF7IP- and EBF1-PDGFRB are constitutively phosphorylated by but do not bind with c-Cbl.

Roles of MAP Kinases and AKT in ATF7IP-PDGFRB-Medicated, IL-3-Independent Proliferation of Ba/F3 Cells

Since ATF7IP-PDGFRB has been shown to phosphorylate MAP kinases and AKT, an examination was made on the effects of MEK and PI3 kinase inhibitors on the IL-3-independent proliferation of the ATF7IP-PDGFRB-expressing Ba/F3 cells. As presented in FIG. 10A, the AKT inhibitor LY294002 induced cell death in both of the ATF7IP-PDGFRB-expressing Ba/F3 cells and the Mock-expressing Ba/F3 cells. Interestingly, the MEK inhibitor PD98059 induced cell death only in the ATF7IP-PDGFRB-expressing Ba/F3 cells, and not in the Mock-expressing Ba/F3 cells. Also, it was confirmed that the simultaneous addition of the conditioned medium containing IL3 partially avoided MEK inhibitor-induced cell death in the ATF7IP-PDGFRB-expressing Ba/F3 cells (FIG. 10B). Further, it was confirmed that treatment with the MEK inhibitor PD98059 markedly reduced the phosphorylation of MAP kinase, as assessed by immunoblotting (FIG. 11). Similarly, treatment with the PI3 kinase inhibitor LY294002 clearly reduced the phosphorylation of AKT (FIG. 11).

Also examined were the kinetics of MAP kinase and AKT phosphorylation mediated by WT-PDGFRB. As shown in FIG. 12, PDGFBB stimulation of the WT-PDGFRB-expressing Ba/F3 cells starved for IL-3 induced the transient but significant phosphorylation of both MAP kinases and AKT, but this phosphorylation diminished in a time-dependent manner even though PDGFBB was left present consistently. In the presence of the conditioned medium containing IL-3, the conditioned medium itself induced the phosphorylation of MAP kinases and AKT, and thus the phosphorylation of both of these proteins was somewhat prolonged after PDGFBB stimulation (FIG. 12).

Effects of TKIs on Survival of Ba/F3 Cells Expressing PDGFRB-Related Chimeric Molecules

Next examined were the effects of TKIs on the IL-3-independent proliferation of the ATF7IP-PDGFRB-expressing B a/F3 cells. When the ATF7IP-PDGFRB-expressing B a/F3 cells were exposed to imatinib or dasatinib, cell proliferation was significantly inhibited in a concentration-dependent manner, and the viable cell number decreased to below 10% after 48-hour incubation with imatinib at a concentration of 25 nM and dasatinib at a concentration of 2.5 nM (FIG. 13). The calculated IC₅₀ value of imatinib against the ATF7IP-PDGFRB-expressing Ba/F3 cells at 24 hours of incubation was 45.6 nM, being much lower than the value of 600 nM estimated for BCR-ABL-expressing Ba/F3 cells in the previous literature (Tomita, et al., Leukemia Research, 2014, 38, 361-370). Also examined were the effects of other next-generation TKIs, including nilotinib, bafetinib, rebastinib, and ponatinib. As shown in FIG. 13, these TKIs effectively reduced the viable cell number of the ATF7IP-PDGFRB-expressing Ba/F3 cells. Similarly, the EBF1-PDGFRB-expressing Ba/F3 cells also exhibited marked sensitivity to the TKIs (FIG. 13). Further, it was confirmed that the phosphorylation of MAP kinases/AKT and PDGFRB actually diminished in the ATF7IP- and EBF1-PDGFRB-expressing Ba/F3 cells treated with TKI, as assessed by immunoblot analysis (FIG. 14).

Discussion

In this example, it was demonstrated that the expression of ATF7IP-PDGFRB induces IL-3-independent proliferation of Ba/F3 cells, which indicates the transforming potential of ATF7IP-PDGFRB, as in the cases of EBF1-PDGFRB and other PDGFRB-related fusion-kinases detected in MPNs (Carroll M., et al., Proc Natl Acad Sci USA, 1996, 93, 14845-14850; Wilbanks A. M., et al., Experimental Hematology, 2000, 28, 584-593; Toffalini F., et al., J Biol Chem, 2010, 285, 12268-12278; Roberts K. G., et al., Cancer Cell, 2012, 22, 153-166). The data obtained in this example show that ATF7IP-PDGFRB is constitutively phosphorylated on tyrosine residues and activates downstream molecules, including AKT, PLC-γ, MAP kinases, p38 MAP kinase, and JNK. Furthermore, the activation of AKT and MAP kinases was shown to be essential for the IL-3-independent survival of Ba/F3 cells. In contrast, the data of this example also clarified that WT-PDGFRB transduced in Ba/F3 cells was not sufficient for the IL-3-independent survival of Ba/F3 cells, while stimulation with PDGFBB partially relieved Ba/F3 cells from the apoptotic cell death induced by the withdrawal of IL-3.

As for the mechanism of the constitutive activation of ATF7IP-PDGFRB, the deregulation of c-Cbl-mediated ubiquitination was believed to be involved in it (Toffalini F., et al., Haematologica, 2009, 94, 1085-1093). It has been well-documented that PDGFRB and other receptor tyrosine kinases activated by binding with ligands phosphorylate and activate c-Cbl promptly, leading to c-Cbl-mediated polyubiquitination of the receptor tyrosine kinases, and that this process is essential for the self-down-regulation of activated growth-factor receptor (Miyake S., et al., J Biol Chem, 1999, 274, 16619-16628; Yokouchi M., et al., J Biol Chem, 1999, 274, 31707-31712). Consistent with previous reports, the marked fragmentation of WT-PDGFRB, but not ATF7IP-PDGFRB or EBF1-PDGFRB, was observed in Ba/F3 transfectants. Also, in Ba/F3 transfectants, there was observed an increased binding of c-Cbl to WT-PDGFRB after stimulation with PDGFBB, but no significant binding of c-Cbl to ATF7IP-PDGFRB or EBF1-PDGFRB was observed. The data suggest that the activation of WT-PDGFRB with ligands is self-limited by induction of c-Cbl-mediated ubiquitination and thus is not enough to fully support IL-3-independent cell survival. In contrast, constitutively activated ATF7IP- and EBF1-PDGFRB phosphorylate c-Cbl protein but do not bind with c-Cbl and, thus, can escape from ubiquitination (Toffalini F., et al., Haematologica, 2009, 94, 1085-1093). Persistent activation of downstream molecules mediated by ATF7IP- and EBF1-PDGFRB may be critical for the IL-3-independent survival of Ba/F3 cells.

The mechanism of the deregulation of c-Cbl-mediated ubiquitination that occur in ATF7IP-PDGFRB may be explained in some ways. For example, Toffalini and co-workers reported that since the TEL-PDGFRB and FIP1L1-PDGFRA fusion proteins, unlike wild-type receptors, are not inserted in the plasma membrane but reside in the cytoplasm, the lack of ubiquitination of these fusion proteins is associated with a difference in their localization (Toffalini F., et al., Haematologica, 2009, 94, 1085-1093). On the other hand, in this example, significant phosphorylation of PLC-γ was observed in both of the ATF7IP- and EBF1-PDGFRB-transduced Ba/F3 cells, but no such phosphorylation was observed in the WT-PDGFRB-transduced cells even if they were stimulated with PDGFBB. Since it has been reported that c-Cbl and PLC-γ compete for the Tyr1021 residue as a docking site and are activated by PDGFRB in a mutually exclusive manner (Reddi A. L., et al., J Biol Chem, 2007, 282, 29336-29347), it is likely that ATF7IP-PDGFRB may preferentially activate PLC-γ and, thus, escape from binding with c-Cbl due to the mutually exclusive effect, avoiding c-Cbl-mediated ubiquitination.

As shown in this example, the PI3 kinase inhibitor leads to cell death in both of the ATF7IP-PDGFRB-transduced cells and the Mock cells maintained by IL-3-containing WEHI-3-conditioned medium, which indicates that signals mediated by AKT are essential for cell survival in both cases. In contrast, the MEK inhibitor leads to cell death in the ATF7IP-PDGFRB-transduced cells but not in the Mock cells, which indicates the difference between the cell survival signals induced by ATF7IP-PDGFRB and those induced by the growth factor, and which suggests that MAP kinase-mediated signals are more critical in ATF7IP-PDGFRB-mediated cell transformation. One possible explanation is that the IL-3 receptor can simultaneously activate a surrogate pathway for cell survival other than the MAP kinase cascade, while PDGFRB can not. Alternatively, since PDGFRB induces not only MAP kinase but also both p38 MAP kinase and JNK which mediate apoptotic signals, MAP kinase-mediated anti-apoptotic signals may be essential for overcoming the apoptotic signals mediated by p38 MAP kinase and JNK in growth factor-independent cell proliferation, while the IL-3 receptor may activate another pathway that regulates the above-mentioned apoptotic signals mediated by p38 MAP kinase and JNK. The reason why the growth factor receptors simultaneously activate both survival signals mediated by MAP kinases and apoptotic signals mediated by p38 MAP kinase and JNK is yet to be clarified (Yu J., et al., J Biol Chem, 2000, 275, 19076-19082), but the reason may be due to an alternate self-regulating mechanism of growth factor receptor activation.

Importantly, the data obtained in this example also demonstrate that the ATF7IP-PDGFRB-expressing Ba/F3 cells show high sensitivity to various TKIs, including imatinib, dasatinib, and other new-generation TKIs. The estimated IC₅₀ value of imatinib is much lower than that of BCR-ABL1. This observation is consistent with the clinical observations of other researchers who reported the effectiveness of imatinib in patients with PDGFRB-rearranged leukemia (Weston B. W., et al., J Clin Oncol, 2013, 31, e413-416; Lengline E., et al., Haematologica, 2013, 98, e146-48; Cheah C. Y., et al., Blood, 2014, 123, 3574-3577). TKIs can effectively inhibit the kinase activity of PDGFRB and reduce the activation of AKT and MAP kinases, which is required for cell survival. The high sensitivity of rearranged PDGFRB kinases to TKIs is important in the therapeutic consideration of the use of TKIs in patients with PDGFRB-rearranged ALL.

As stated above, ATF7IP-PDGFRB and EBF1-PDGFRB have so far been found only in ALL patients, but other PDGFRB fusion genes have been found only in myeloid neoplasms, which suggests selectivity between chimeric partners of PDGFRB and the lineage of the developing neoplasms, though the precise mechanism of this selectivity is yet to be clarified. Interestingly, Tomasson and co-workers reported that, in a murine BMT model, the transduction of TEL-PDGFRB into the whole bone marrow caused a rapidly fatal myeloproliferative disease, whereas phenylalanine mutants with tyrosine residues 579/581 in the fusion gene developed T-cell lymphomas (Tomasson M. H., et al., J Clin Invest, 2000, 105, 423-432). The difference in chimeric partner may give rise to a difference in conformation of the PDGFRB kinase domain which affects the lineage commitment of the developing neoplasms. An elucidation of the mechanism of the selectivity of ATF7IP-PDGFRB for ALL development is awaited.

In conclusion, ATF7IP-PDGFRB has a transforming potential and exhibits significant sensitivity to TKIs as compared to BCR-ABL1. The observations obtained in this example are consistent with the clinical findings, and indicate the therapeutic benefit of TKIs for a subset of BCP-ALL harboring PDGFRB-related chimeric kinases. In addition, further clarification of the molecular basis of chimeric PDGFRB kinase-mediated cell transformation should provide a new therapeutic strategy against hematologic malignancies caused by PDGFRB translocation.

### INDUSTRIAL APPLICABILITY

The present invention makes it possible to detect a gene fusion newly discovered as a responsible mutation for cancer; to identify patients with cancer or subjects with a risk of cancer, in whom a substance suppressing the expression and/or activity of a polypeptide encoded by a fusion polynucleotide produced by said gene fusion shows a therapeutic effect; and to provide suitable treatment (personalized medicine) for such cancer patients.

### SEQUENCE LISTING FREE TEXT

SEQ ID NOs: 1 and 3: ATF7IP-PDGFRB fusion polynucleotides
SEQ ID NOs: 2 and 4: ATF7IP-PDGFRB fusion polypeptides
SEQ ID NOs: 5 and 7: ATF7IP cDNAs
SEQ ID NOs: 6 and 8: ATF7IP polypeptides
SEQ ID NO: 9: PDGFRB cDNA
SEQ ID NO: 10: PDGFRB polypeptide
SEQ ID NO: 11: Forward primer
SEQ ID NO: 12: Reverse primer
SEQ ID NO: 13: ATF7IP-1 Fwd
SEQ ID NO: 14: ATF7IP-1 Rev
SEQ ID NO: 15: ATF7IP-2 Fwd
SEQ ID NO: 16: ATF7IP-2 Rev
SEQ ID NO: 17: ATF7IP-PDGFRB-3 Fwd
SEQ ID NO: 18: ATF7IP-PDGFRB-3 Rev
SEQ ID NO: 19: EBF1-PDGFRB Fwd
SEQ ID NO: 20: EBF1-PDGFRB Rev
SEQ ID NO: 21: ATF7IP-PDGFRB detection Fwd-1
SEQ ID NO: 22: ATF7IP-PDGFRB detection Rev-1
SEQ ID NO: 23: EBF1-PDGFRB detection Fwd-1
SEQ ID NO: 24: EBF1-PDGFRB detection Rev-1
SEQ ID NO: 25: WT-PDGFRB detection Fwd-1
SEQ ID NO: 26: WT-PDGFRB detection Rev-1

1. A method for detecting a gene fusion, the method comprising the step of detecting an ATF7IP-PDGFRB fusion polypeptide comprising the SETDB1-binding domain of ATF7IP and the transmembrane region and kinase domain of PDGFRB, and having kinase activity, or a fusion polynucleotide encoding said polypeptide, in an isolated sample from a subject.

2. The method according to claim 1, wherein the fusion polypeptide is any one of (i) to (iii) mentioned below:
(i) a polypeptide consisting of the amino acid sequence of SEQ ID NO: 2 or 4,
(ii) a polypeptide consisting of an amino acid sequence derived from the amino acid sequence of SEQ ID NO: 2 or 4 by deletion, substitution or addition of one or more amino acids, and the polypeptide having kinase activity, or
(iii) a polypeptide consisting of an amino acid sequence having a sequence identity of at least 80% to the amino acid sequence of SEQ ID NO: 2 or 4, and the polypeptide having kinase activity.

3. The method according to claim 1, wherein the fusion polynucleotide is any one of (i) to (iv) mentioned below:
(i) a polynucleotide consisting of the nucleotide sequence of SEQ ID NO: 1 or 3,
(ii) a polynucleotide that hybridizes under stringent conditions with a polynucleotide consisting of a nucleotide sequence complementary to the polynucleotide consisting of the nucleotide sequence of SEQ ID NO: 1 or 3, and which encodes a polypeptide having kinase activity,
(iii) a polynucleotide that consists of a nucleotide sequence derived from the nucleotide sequence of SEQ ID NO: 1 or 3 by deletion, substitution or addition of one or more nucleotides, and which encodes a polypeptide having kinase activity, or
(iv) a polynucleotide that has a sequence identity of at least 80% to the polynucleotide consisting of the nucleotide sequence of SEQ ID NO: 1 or 3, and which encodes a polypeptide having kinase activity.

4. The method according to claim 1, wherein the gene fusion is a responsible mutation (driver mutation) for cancer.

5. The method according to claim 4, wherein the cancer is acute lymphoblastic leukemia.

6. A method for identifying a patient with cancer or a subject with a risk of cancer, in whom a substance suppressing the expression and/or activity of a polypeptide encoded by a fusion polynucleotide produced by a gene fusion serving as a responsible mutation (driver mutation) for cancer shows a therapeutic effect, the method comprising the steps of:
(1) detecting an ATF7IP-PDGFRB fusion polypeptide comprising the SETDB1-binding domain of ATF7IP and the transmembrane region and kinase domain of PDGFRB, and having kinase activity, or a fusion polynucleotide encoding said polypeptide, in an isolated sample from a subject, and
(2) determining that the substance suppressing the expression and/or activity of the polypeptide shows a therapeutic effect in the subject, in the case where the fusion polypeptide or the fusion polynucleotide encoding said polypeptide is detected.

7. A kit for detecting a gene fusion, the kit comprising any or a combination of (A) to (C) mentioned below:
(A) a polynucleotide that serves as a probe designed to specifically recognize an ATF7IP-PDGFRB fusion polynucleotide;
(B) polynucleotides that serve as a pair of primers designed to enable specific amplification of an ATF7IP-PDGFRB fusion polynucleotide; or
(C) an antibody that specifically recognizes an ATF7IP-PDGFRB fusion polypeptide.

8. The kit according to claim 7, wherein the gene fusion is a responsible mutation (driver mutation) for cancer.

9. An isolated ATF7IP-PDGFRB fusion polypeptide or a fragment thereof, which comprises the SETDB1-binding domain of ATF7IP and the transmembrane region and kinase domain of PDGFRB, and has kinase activity.

10. A polynucleotide encoding the fusion polypeptide or the fragment thereof according to claim 9.

11. A method for treating ATF7IP-PDGFRB gene fusion-positive cancer, comprising administering, to a patient in need thereof, an effective amount of a substance suppressing the expression and/or activity of an ATF7IP-PDGFRB fusion polypeptide comprising the SETDB1-binding domain of ATF7IP and the transmembrane region and kinase domain of PDGFRB, and having kinase activity.

12. (canceled)

13. The method according to claim 11, wherein the cancer is acute lymphoblastic leukemia.

14. The method according to claim 11, wherein the substance suppressing the expression and/or activity of an ATF7IP-PDGFRB fusion polypeptide comprising the SETDB1-binding domain of ATF7IP and the transmembrane region and kinase domain of PDGFRB, and having kinase activity is a substance inhibiting the kinase activity of PDGFRB.

15. The method according to claim 14, wherein the substance inhibiting the kinase activity of PDGFRB is imatinib mesylate, dasatinib, nilotinib, ponatinib, rebastinib or bafetinib.

16. The method according to claim 14, wherein the substance inhibiting the kinase activity of PDGFRB is dasatinib.

17. A method for screening a cancer therapeutic agent, the method comprising the steps of:
(1) bringing a test substance into contact with a cell that expresses an ATF7IP-PDGFRB fusion polypeptide comprising the SETDB1-binding domain of ATF7IP and the transmembrane region and kinase domain of PDGFRB, and having kinase activity;
(2) determining whether the expression and/or activity of the fusion polypeptide is suppressed or not; and
(3) selecting the substance determined to suppress the expression and/or activity of the fusion polypeptide, as a cancer therapeutic agent.
